(19) United States
(12) Patent Application Publication
(10) Pub. No.: US 2004/0097973 A1
(43) Pub. Date: May 20, 2004

(54) Transvascular bybass method and system

(76) Inventors: Amir Loshakove, Moshav-Bazra (IL); Ofer Nativ, Rishon-Lezion (IL); Ido Kilemnik, Herzlia (IL); Tanchum Feld, Izrael (IL); Amnon Yadin, Pleasanton, CA (US)

Correspondence Address: William H Dippert, Reed Smith, 29th Floor, 599 Lexington Avenue, New York, NY, 10022-7650, US

(21) Appl. No.: 10/239,364

(22) PCT Filed: Mar. 20, 2001
(86) PCT No.: PCT/IL01/00267

(30) Foreign Application Priority Data
Mar. 20, 2000 (IB) 00/00302
Mar. 20, 2000 (IB) 00/00310
Sep. 28, 2000 (IL) 00/00609
Sep. 28, 2000 (IL) 00/00611
Jan. 25, 2001 (IL) 01/00074

(51) Int. Cl. A61B 17/04

(52) U.S. Cl. 606/144

(57) ABSTRACT

An anastomosis delivery system for delivering a connector having at least one backwards spike having a bent tip, comprising: a hollow guide sheath; and a hollow, axially slotted section, fitting within said sheath, said section having a flared configuration and an unflared configuration and wherein said axially slotted section is adapted to contain at least a part of said connector and to limit axial motion of said connector when said section is in its unflared configuration.

### RELATED APPLICATIONS

This application is related to PCT publications and applications WO99/62415, WO00/56226, WO00/56227, PCT/IL00/00611, WO00/56228, PCT/IL00/00609 and PCT/IL01/00074, all of which designate the US, the disclosures of which are incorporated herein by reference. This application also claims the benefit under 119 (e) of 60/254,689, the disclosure of which is incorporated herein by reference. This application is also related to an application titled “GRAFT AND CONNECTOR DELIVERY”, filed on even date by same applicant in the Israel receiving office of the PCT, the disclosure of which is incorporated herein by reference.

The present invention relates to performing anastomotic connections, for example, via a vascular system.

### BACKGROUND OF THE INVENTION

Bypass procedures, in which a clogged vessel, for example in the heart, is bypassed by an unclogged conduit, are well known in the art. Recently, the desirability of performing this procedure using a vascular approach, has come to prominence, at least because the surgical wound is less traumatic to the patient. This procedure is known as a transvascular procedure.

In a transvascular procedure, however, there is a danger that the various tools and devices, which are provided through a catheter, will be damaged by or damage the catheter and/or be deployed incorrectly.

A competing method is operating through a small hole in the chest, a mini-thoractomy. However, this method cannot generally be used where there are more than two vessels to bypass, as is often the case.

### SUMMARY OF THE INVENTION

An aspect of some embodiments of the invention relates to protecting a delivery catheter and tools being delivered via the catheter during a bypass procedure. In an exemplary embodiment of the invention, a protective sheath is provided for enclosing a punch, prior to and/or after the punch transfixes the tissue to be punched. Alternatively or additionally, a same or different protective sheath is provided for enclosing and, optionally assisting in deployment, of an anastomotic connector.

Alternatively, in an exemplary embodiment of the invention, an outer cutting tube of a punch is used as the protective sheath for the punch.

Optionally, the sheath is more rigid at its distal end, where it protects the tool.

Optionally, the sheath is shaped to aim the tools to be perpendicular (or at any other desired angle) to the wall of the blood vessel from which the procedure is performed, for example, an aorta

An aspect of some embodiments of the invention relates to a guide for deployment of an anastomotic device. In an exemplary embodiment of the invention, the guide comprises a plurality of receptacles for maintaining bent back spikes of an anastomotic connector in a radially compressed and/or pulled back position. In an exemplary embodiment of the invention, the tips of the spikes are bent, even if the body of the spike is straightened for delivery. Optionally, the guide prevents the connector from pulling itself out prematurely, for example, if front spikes of the connector engage nearby tissue. Optionally, the guide also restrins front spikes of the connector. In some embodiments, the receptacle comprises an inner lip in the guide, possibly allowing the connector some axial motion, until the back spikes hit the lip. This allows the front spikes of the connector to exit and engage nearby tissue, without pulling the whole connector out of the guide. Alternatively, the receptacle comprises holes for holding the tips of the spikes. Optionally, the receptacle comprises a capsule that is closed at one end. In an exemplary embodiment of the invention, the spikes comprise 3, 4, 5, 6 or a greater or fewer number of spikes.

In an exemplary embodiment of the invention, the guide includes a flaring out section distal of the receptacles.

Only when the guide exits a hole in an aorta, the flaring out portion spreads out, freeing the back spikes to engage the aorta A similar mechanism may be used for entering a blood vessel, for example a coronary vessel, in which the flaring out occurs inside the free volume of the vessel, freeing back and/or front spikes of the connector. In an exemplary embodiment of the invention, the flaring out portion comprises a tube with axial splits. Possibly, a balloon or other expanding device is used to force the flaring. Alternatively, the tube may be pre-stressed to flare out when released.

Alternatively, the bent part of the spike is held between two elements such as tubes and/or elongate members. In one exemplary embodiment of the invention, the two elements define at their tip a receptacle for the bent spike tips (e.g., perpendicular to the guide axis). Alternatively, the two elements hold the spike by radial pressure. Optionally, at least one of the elements includes a slot or window for receiving the bent portion of the back spike.

In an exemplary embodiment of the invention, the guide comprises a capsule with one closed end. Optionally, the connector is held by inserting an inner mandrel (or object, such as a bead) between the backward spikes.

An aspect of some embodiments of the invention is an anti-dislodgement mechanism for a catheter tip that is inserted into (and/or out of) a hollow organ, for example a blood vessel, through an entry hole. In an exemplary embodiment of the invention, the catheter, at least at its tip, includes two layers connected at their tips, namely an inner tube and an outer, axially slit tube. When the inner tube is retracted concurrently with maintaining the outer tube in place, the slit portion of the outer tube flares out to have a diameter greater than that of the entry hole, for example, twice or three times the radius, so that the catheter cannot be retracted.

An aspect of some embodiments of the invention relates to a guided punch. In an exemplary embodiment of the invention, a hole is punched in a vessel, for example an aorta, by penetrating the aorta with a thin guide wire and then advancing the punch over the guide wire. Optionally, an intermediate thickness tube is advanced into the hole formed by the guide wire, prior to advancing the punch. Optionally, the intermediate tube has a blunt end and is used to enclose the tip of the guide wire and prevent inadvertent puncturing of other body tissues. Optionally, the guide wire is retracted after it is used to penetrate the aorta, so that only the less sharp objects (e.g., the punch tip) are extended. The punch may be, for example, a rotating cutting punch or a axially moving punch.

An aspect of some embodiments of the invention relates to a rotating punch mechanism. In an exemplary embodiment of the invention, the punch comprises a central guide portion and a surrounding outer cutting tube. An inner diameter of the cutting tube defines the diameter of the cut. In an exemplary embodiment of the invention, the central guide portion, for example, a thin guide-wire like portion, is inserted into the target tissue to be punched Possibly, the central guide portion includes a stop to prevent over-penetration of the guide portion. The cutting tube is then pushed against the target tissue and rotated around the guide portion to cut out a section of the tissue. Optionally, the outer tube is coupled to the central guide, so that it is advanced with it. Alternatively or additionally, the outer tube is elastically urged against the target tissue. Alternatively or additionally, the outer tube is manually advanced.

Optionally, the cutting tube advances as it rotates, for example, on a screw. Optionally, he advance is limited to a fixed amount, for example, to be less or somewhat more than the thickness of the punched vessel, for example, between 3 mm and 9 mm for an aorta.

An aspect of some embodiments of the invention relates to an anastomosis connector having a plurality of non-penetrating spikes, each of which is formed by the meeting, at an angle, of two arms. Optionally, the plurality of spikes is merged into a single unit In an exemplary embodiment of the invention, the connector comprises a cylindrical or ring body 5 having, at one end thereof, a plurality of non-penetrating spikes. In an exemplary embodiment of the invention, the spikes are merged into an undulating curve, curved areas of which act as the spike parts in contact with vascular tissue. In an exemplary embodiment of the invention, the curve serves to apply pressure to a wall of a blood vessel (e.g., an aorta), that is perpendicular to the central axis of the connector. Optionally, the spikes are designed to bend (e.g., by locally weakening the connector) or are pre-bent at at least two locations. One bend location causes part of the curve to lie perpendicular to the cylinder axis. A second bend location causes the rest of the curve to lie at a sharp angle to the cylinder axis. In an exemplary embodiment of the invention, the spikes are curved in the bending plane so that they can better apply pressure to a perpendicular blood vessel wall.

In an exemplary embodiment of the invention, the curve defines areas of higher curvature, which areas twist when the spikes are deployed. Alternatively, a torsion bar is provided at points of high twisting. Alternatively or additionally, two or more torsion bars and/or torsion joints are provided in series. In one example, a spike is bent 180° by providing two torsion bars or joints, one for each bend. In an exemplary embodiment of the invention, each torsion area is defined by two arms that define the ends of the bar. In an exemplary embodiment of the invention, the spike comprises two arms that meet a torsion bar and two more arms extend from the torsion bar, and meet at a second torsion bar. One or more arms extending from the second bar define the tip of the spike (or another torsion bar). Alternatively, a torsion bar or area is defined between two arms that meet at an angle or at a slight offset (e.g., with the twist area being defined in the offset).

An aspect of some embodiments of the invention relates to loading of an anastomosis connector into a delivery system used for a vascular approach. In one example, the delivery system comprises a tube that encloses at least part of the connector. In an exemplary embodiment of the invention, the connector has a set of forward pointing spikes and a set of backwards pointing spikes and the connector is mounted by bending back the backwards set of spikes and restraining the backwards spikes in the delivery system. Optionally, however, the bent tips of the backwards spikes remain bent. The forward spikes are optionally not bent backwards, for example being restrained by the delivery system or sticking out of the delivery system.

In an exemplary embodiment of the invention, the backwards spikes are bent back by enclosing each spike in a flexible tube and pulling the tubes through the delivery system. Alternatively, the spikes are bent back with a tool that bends the spikes back to fit into tube of the delivery system.

In an exemplary embodiment of the invention, the connector is held, in the delivery system, between an inner and an outer tube. In an exemplary embodiment of the invention, the connector is held using a pre-defined bend in the backwards spikes of the connector. In an exemplary embodiment of the invention, the inner and outer tube define a step that engages the bent tip of the spikes. Alternatively or additionally, the inner tube defines a slot that receives the bend area itself.

An aspect of some embodiments of the invention relates to the injection of contrast material during a bypass procedure. In an exemplary embodiment of the invention, a catheter is provided in an aorta or other large vessel and then exits the vessel to perform a bypass. In an exemplary embodiment of the invention, the catheter comprises a sheath, optionally bent to lay perpendicular to the aorta, and an inner punch mechanism. Optionally, the punch mechanism includes an inner sheath. Optionally, the punch mechanism is replaced by a graft delivery system. In an exemplary embodiment of the invention, injection of contrast material is used to determine that the catheter is near the aorta wall. In an exemplary embodiment of the invention, the catheter is aimed so that when it exits the aorta, it will enter fatty tissue rather than cardiac tissue. Imaging may be, for example, using X-ray fluoroscopy, CT or open MRI.

Alternatively or additionally, contrast material is injected outside the aorta In an exemplary embodiment of the invention, the thickness of the aorta is measured by imaging the area and measuring the distance between different areas with contrast material. Alternatively or additionally, the external contrast material is used as a landmark for determining how far to advance the punch, graft and/or a connector on the graft. Alternatively or additionally, contrast material is injected into the graft, from the aorta, to detect leaks.

In an exemplary embodiment of the invention, the catheter system includes multiple ports for contrast material (e.g., in the catheter handle), including: in the sheath (outside of the punch), in the punch and optionally in the inner sheath of the punch. Optionally, one or more dedicated contrast material channels are provide din the catheter, for example, as separate tubes.

An aspect of some embodiments of the invention relates to utilizing the venous coronary system for providing arterial blood to the heart. In an exemplary embodiment of the invention, the coronary sinus is blocked and the coronary sinus and/or one of the veins leading to it are connected, possibly via a bypass conduit, to the arterial system, for example to the aorta or to a mammary artery. It is expected that the veins will provide blood to the heart, possibly becoming more artery-like as time goes on. Optionally, one of the veins is disconnected from the coronary sinus and connected, possibly via a bypass conduit, to the vena cava or another part of the venous system, to provide drainage from the coronary vascular system.

There is thus provided in accordance with an exemplary embodiment of the invention, an anastomosis delivery system for delivering a connector having at least one backwards spike having a bent tip, comprising:

a hollow guide sheath; and

a hollow, axially slotted section, fitting within said sheath, said section having a flared configuration and an unflared configuration and wherein said axially slotted section is adapted to contain at least a part of said connector and to limit axial motion of said connector when said section is in its unflared configuration. Optionally, axially moving said section selectively advances said spike. Alternatively or additionally, axially moving said section selectively retracts said spike.

In an exemplary embodiment of the invention, said slotted section maintains said bent tip in a bent configuration.

In an exemplary embodiment of the invention, said slotted section includes at least one receptacle for engaging said bent tip. Optionally, said receptacle comprises an inner lip of said section, adapted for catching said tip. Alternatively or additionally, said receptacle comprises a hole in said section, for engaging said tip.

In an exemplary embodiment of the invention, said section comprises a second, inner tube and wherein said inner tube and said slotted section define between them a receptacle for a bent section of at least one bent spike of connector. Optionally, said receptacle is a space between tips of said slotted section and said inner tube.

In an exemplary embodiment of the invention, said receptacle is an opening in said inner tube. Alternatively or additionally, said slotted section and said inner tube grip between them a part of said connector.

In an exemplary embodiment of the invention, said slotted section comprises a capsule closed at one end.

There is also provided in accordance with an exemplary embodiment of the invention, an anastomosis delivery system for delivering a connector having at least one backwards spike having a bent tip, comprising:

a hollow guide sheath;

an apertured inner tube fitting within said sheath; and

a plurality of spike locking elements disposed between said guide sheath and said apertured inner tube, wherein said spike locking elements, when extended, are adapted to grip a part of said anastomosis connector between said inner tube and said locking elements and wherein said apertures are each adapted to receive a said bent tip of said anastomosis connector.

There is also provided in accordance with an exemplary embodiment of the invention, an anastomosis delivery system for delivering a connector having at least one backwards spike having a bent tip, comprising:

a hollow guide sheath;

a cylindrical capsule having one open end an one closed end; and

an anastomosis connector held in said capsule. Optionally, the system comprises a stopper arranged between a plurality of said backwards spikes and urging said spikes towards said capsule

There is also provided in accordance with an exemplary embodiment of the invention, a method of mounting an anastomosis connector having a plurality of bent backwards spikes including bent tips, into a delivery tube, comprising:

bending back said spikes to point backwards along an axial direction of said connector, away from a graft mounted on said connector;

maintaining said tips in a bent configuration; and

inserting said spikes into a receptacle of said delivery tube, which receptacle maintains said tips in a bent configuration.

Optionally, bending back comprises:

mounting a thin flexible tube on each of said spikes;

threading said tube through a plurality of tip holding apertures in said receptacle; and

retracting said tubes to bend said spikes and pull them into said receptacle. Optionally, the method comprises:

locking said connector in place; and

retracting said tubes to remove them from said spikes.

Additionally, bending back comprises:

pushing back each spike, using a jig, into said receptacle; and

locking said spike tip in said receptacle.

There is also provided in accordance with an exemplary embodiment of the invention, a guided punch, comprising:

a sharp, extendible guide wire; and

a hollow punch mechanism adapted to ride on the guide wire, wherein said guide wire is adapted to extend from said punch. Optionally, said guide wire has a limited extension distance of less than 3 cm. Optionally, said distance is shorter than 1 cm. Optionally, said distance is greater than 0.3 cm.

In an exemplary embodiment of the invention, said punch comprises a hollow tube adapted to fit between said punch mechanism and said guide wire.

In an exemplary embodiment of the invention, said punch is a rotating punch

In an exemplary embodiment of the invention, said punch is an axially moving punch.

In an exemplary embodiment of the invention, said punch is adapted for injection of contrast material inside of said hollow of said punch mechanism.

There is also provided in accordance with an exemplary embodiment of the invention, a rotating punch, comprising:

a sharp, central guide wire; and

a rotating outer tube having a vascular cutting edge defined by a lip of said tube. Optionally, said outer tube advances as it is rotated. Optionally, said advancing is limited to less than 3 cm. Optionally, said advancing is limited to less than 1 cm.

In an exemplary embodiment of the invention, said punch is adapted for a particular target vessel, by matching said advancing limitation to the target vessel.

In an exemplary embodiment of the invention, said cutting edge is smooth. Alternatively, said cutting edge is serrated.

In an exemplary embodiment of the invention, said guide wire is smooth. Alternatively, said guide wire is adapted to engage vascular tissue it is inserted into.

In an exemplary embodiment of the invention, the punch comprises a hollow tube adapted to be brought over said guide wire and within said rotating outer tube. Optionally, said punch is adapted for injection of contrast material inside of said hollow tube.

In an exemplary embodiment of the invention, said punch is adapted for injection of contrast material between said spike and said outer tube.

In an exemplary embodiment of the invention, said outer tube is bent at a right angle, such that positioning perpendicular to a vessel wall is assisted. Alternatively or additionally, said outer tube has an increasing outer diameter, away from said cutting edge.

In an exemplary embodiment of the invention, the punch comprises a balloon distal from said cutting edge, said balloon, when inflated, having an outer diameter slightly greater than a diameter of said outer tube and about the inner diameter of a sheath associated with said punch.

There is also provided in accordance with an exemplary embodiment of the invention, an advancing rotating punch, comprising:

a sharp, central guide wire; and

a rotating outer tube adapted to cut a target vessel which advances relative to said wire when it rotates.

There is also provided in accordance with an exemplary embodiment of the invention, a catheter system, comprising:

an outside sheath having an inner volume;

a first contrast injection port communicating with the inner volume of said sheath;

at least one inner mechanism conveyed by said sheath and having an inner volume; and

a second contrast injection port communicating with the inner volume of said inner mechanism. Optionally, said at least one inner mechanism comprises two switchable inner mechanisms. Alternatively or additionally, said at least one inner mechanism comprises an inner tube and said system comprises a third contrast injection port associated with said inner tube. Alternatively or additionally, said sheath is bent to facilitate perpendicular positioning of a tip of said sheath against an inner wall of a target blood vessel. Optionally, inner mechanism is bent to match said bend in said sheath. Alternatively or additionally, said system comprises a straight guide wire adapted to fit in said sheath and maintain said sheath straight when said sheath is guided to a target area.

In an exemplary embodiment of the invention, said at least one inner mechanism comprises a punch. Optionally, said system comprises an inner tube having a diameter that varies, along its length between a diameter of said punch and an inner diameter of said sheath.

In an exemplary embodiment of the invention, said system comprises balloon distal of said punch and having a diameter that varies between a diameter of said punch and an inner diameter of said sheath.

There is also provided in accordance with an exemplary embodiment of the invention, an anastomotic connector, comprising:

a cylinder-like body, and

at least one set of spikes, coupled to said body by twisting joints. Optionally, said spikes are adapted not to penetrate tissue which the spikes contact. Optionally, said twisting joints comprise at least one torsion bar. Alternatively or additionally, said twisting joints comprise at least one bend area. Alternatively or additionally, said set of spikes are bent. Optionally, said set of spikes are bent at two different locations along the spikes. Alternatively or additionally, each spike comprises two arms that meet at a tip of the spike and are each attached to a different part of said connector. Optionally, each arm is attached to a base extension of said connector, by a twisting joint. Optionally, said arms and said base extensions define a continuous curve.

There is thus provided in accordance with an exemplary embodiment of the invention, a fixating guide sheath for insertion into a blood vessel, comprising:

an inner tube; and

an outer tube, slotted near an end thereof, wherein said inner tube is retracted relative to said outer tube, said slotted outer tube flares out to prevent further retraction of said sheath. Optionally, said sheath is bent near said end.

### BRIEF DESCRIPTION OF TEE DRAWINGS

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements or parts which appear in more than one figure are preferably labeled with a same or similar number in all the figures in which they appear, in which:

FIGS. 1-15 illustrate a process of performing a proximal transvascular anastomosis, in accordance with an exemplary embodiment of the invention;

FIG. 16 illustrates a capsule for guiding the delivery of an anastomosis connector, in accordance with an exemplary embodiment of the invention;

FIG. 17 illustrates an alternative catheter delivery system, including a separate protective sheath, in accordance with an exemplary embodiment of the invention;

FIGS. 18-22 illustrate a guided punch, in accordance with an exemplary embodiment of the invention;

FIGS. 23A and 23B illustrate an anti-dislodgment mechanism for a catheter, in accordance with an exemplary embodiment of the invention;

FIG. 24A illustrates a rotating and cutting out punch mechanism, in accordance with an exemplary embodiment of the invention;

FIGS. 24B-24D show an exemplary rotating punch, in accordance with an exemplary embodiment of the invention;

FIGS. 24E-24F show an alternative rotating punch, in accordance with an exemplary embodiment of the invention;

FIG. 25 illustrates a device delivery guide, as an alternative to the capsule shown in FIG. 16, in accordance with an exemplary embodiment of the invention;

FIG. 26 is an exploded view of the guide system of FIG. 25;

FIGS. 27A-27C illustrate two exemplary anastomosis connectors, in accordance with an exemplary embodiment of the invention;

FIGS. 28A-28B illustrate a method of mounting a connector, such as the connector of FIG. 27, into a delivery system, in accordance with an exemplary embodiment of the invention;

FIGS. 29A-29D show a method of mounting a connector, in accordance with an alternative exemplary embodiment of the invention; and

FIGS. 30A-30C show details of the process of attaching the connector of FIG. 27 to an aorta, in accordance with an exemplary embodiment of the invention

### DETAILED DESCRIPTION OF EXEMPLARY EMBODIMENTS

In a transvascular procedure at least part of the procedure is performed via a catheter. In one example, the provision of a graft and/or its attachment to a source artery are performed via a catheter. The other side of the anastomosis, for example, may be performed via the same or a different catheter and via a same or different vessel or it may be performed using a more invasive technique, such as open surgery or (mini-) thoractomy. In an exemplary embodiment of the invention, the transvascular technique is used to provide grafts for multiple bypass operations, with one or more mini-thoractomy openings being used to attach the grafts to target coronary vessels.

Although the following description focuses on the heart, the following devices and/or procedures may be used for other organs and bypass procedures as well, as appropriate.

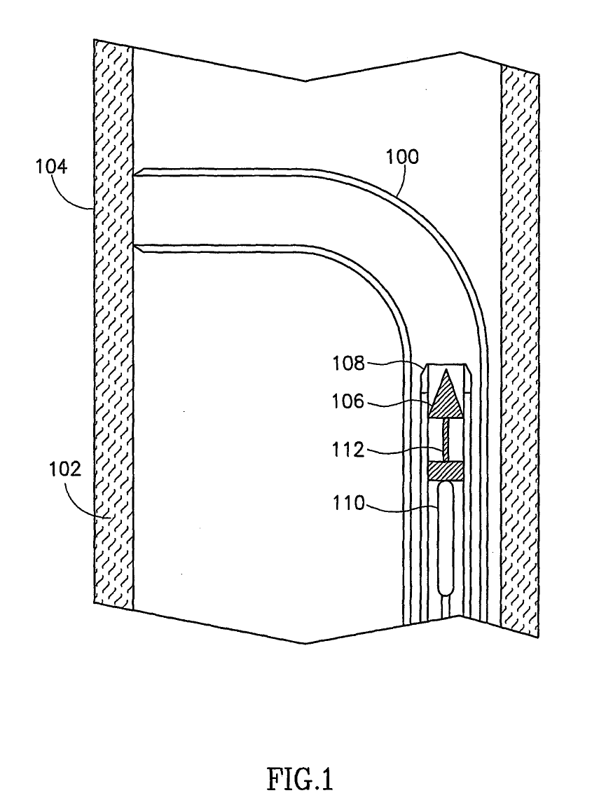

FIGS. 1-15 illustrate a process of performing a proximal transvascular anastomosis, in accordance with an exemplary embodiment of the invention. In this process, a catheter is brought against the inside of an aortic wall, a hole is punched out of an aorta, the catheter is advanced into the punched out hole, an anastomosis connector mounted on a graft is positioned in the hole and then the catheter is retracted and the connector is deployed.

In an exemplary embodiment of the invention, catheter 100 is a J-tip catheter. Optionally, a rigid stylet is used for insertion and/or navigation of the catheter.

FIG. 1 shows a guiding catheter 100, being brought against an inside wall of an aorta 102 at a location 104 thereof. A punch mechanism provided inside catheter 100 includes a needle punch 106 having a punch area 112 adapted to receive tissue to be punched out and an outer punch tube 108 which cooperates with needle punch 106 to cut off the received tissue. Optionally, a balloon 110 is provided proximal of needle punch 106. Its use, and that of an alternative mechanism, will be described below. Catheter 100 may include a hemostat valve, to prevent blood leakage.

As shown in FIG. 1, during feeding of the punch mechanism, outer tube 108 is optionally brought forward (or needle punch 106 kept retracted relative to the outer tube) over the tip of needle punch 106, to prevent the tip from inadvertently engaging catheter 100, aorta 102 and/or other nearby tissues or devices.

In an exemplary embodiment of the invention, catheter 100 includes a bend, to support correct angular orientation to the aorta wall. Optionally, the punch includes a matching bend. In an exemplary embodiment of the invention, the catheter is inserted in a straight manner and when a guide wire or stylet is removed from the catheter, it reverts to its bent orientation. Contrast material may also be injected before the stylet is removed, to allow the position of catheter 100 to be determined. In an exemplary embodiment of the invention, the catheter is oriented in a direction that ensures that there is no critical and/or sensitive tissue right outside the aorta, where it might be damaged by the bypass procedure.

In an exemplary embodiment of the invention, contrast material (e.g., x-ray, CT, MRI or ultrasound contrast material) is injected through catheter 100 to ensure that its tip contacts the wall if the catheter is close enough to the wall, the profile of the wall and of the catheter are expected to show up in the image. It should be noted that due to the fast flow in the aorta, it may be desirable to time the imaging to the provision of the contrast.

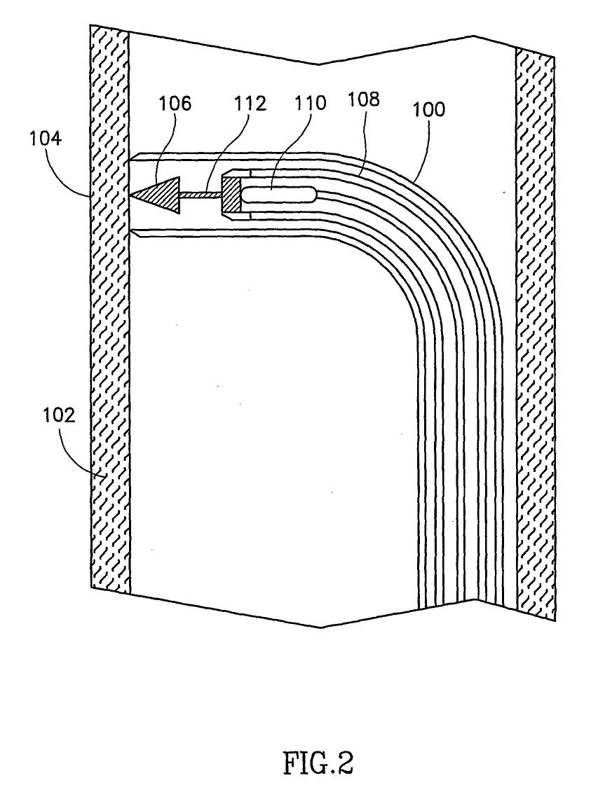

In FIG. 2, needle punch 106 is brought up against location 104 and outer tube 108 is retracted.

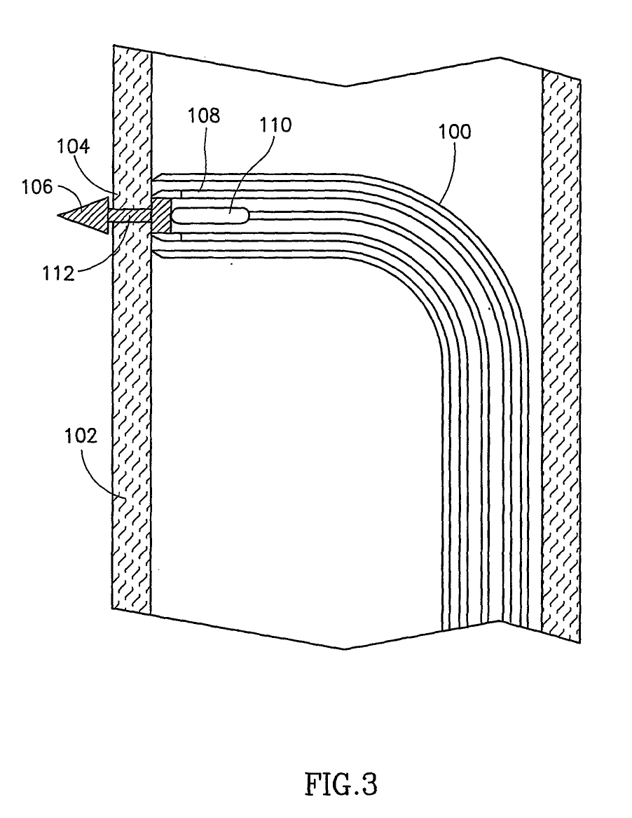

In FIG. 3, needle punch 106 is advanced through aorta 102, so that the wall of the aorta is received in punch area 112. Optionally, this penetration is sensed (manually) or seen, for example by injecting contrast material into catheter 100 and viewing the relative location of punch 106 and the wall.

As described below, punch 106 may comprise a sharp tip that once inserted is replaced by or covered by an over tube that is less sharp. In an exemplary embodiment of the invention, contrast material is injected out of the aorta through the punch or through the sheath, to ensure the punch is outside the aorta. Alternatively or additionally, contrast material is injected between the sheath and the punch. Comparing the two sets of injections allows a determination of the thickness of the aorta wall.

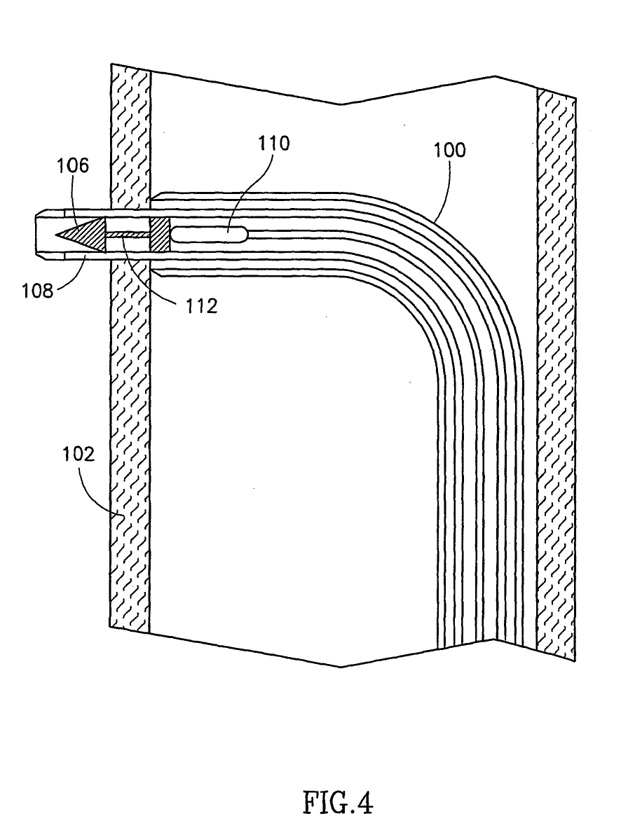

In FIG. 4, outer tube 108 is advanced through aorta 102 and past punch area 112, where it cuts out the received portion of the aorta. Optionally, outer tube 108 is advanced past the tip of needle punch 106, to protect tissue outside the blood vessel (or inside, for inward punching) from being damaged by the tip. In an inward punching embodiment, it is the blood vessel wall, away from the punch location that is protected. Optionally, the motions of needle punch. 106 and outer tube 108 are coupled so that a user needs to operate only a single control. In one example, the advance of needle punch 106 a certain distance (e.g., through the aorta), releases a spring loaded mechanism that advances outer tube 108 past the tip of needle punch 106. Alternatively, a less automatic mechanisms may be used, for example one in which stops are provided in the controls, so that manual motion of the needle punch and/or the outer sleeve is stopped by the stop when a desired relative position is achieved. Alternatively or additionally, suitable markings for the different tubes are provided in the part of the delivery system outside the body. In one example, the handle of catheter 100 and/or the proximal end of outer tube 108 are transparent or slotted, so the relative locations of the needle punch tube (its proximal end) and/or the outer tube, can be seen. Such mechanisms may optionally be used for the methods shown in the other figures.

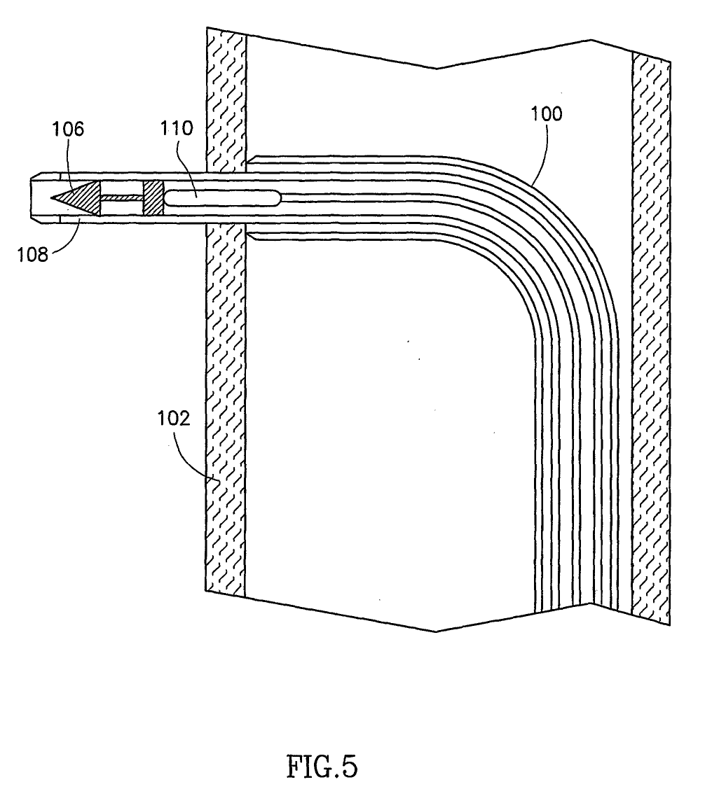

In FIG. 5, balloon 110 is positioned to be inside the hole in the aorta. This is an optional procedure, used to assist in inserting the catheter 100 into the hole in the aorta. Balloon 110 may be fixed to needle punch 106. Alternatively, it may be conveyed over the length of the proximal part of needle punch 106.

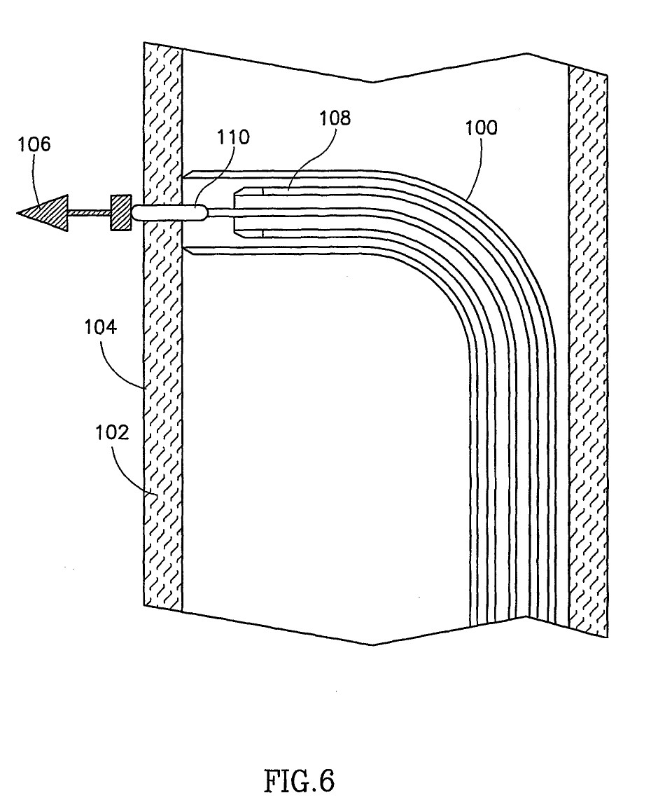

In FIG. 6, outer tube 108 is retracted, leaving balloon 110 in contact with the aorta, sealing the hole in the aorta.

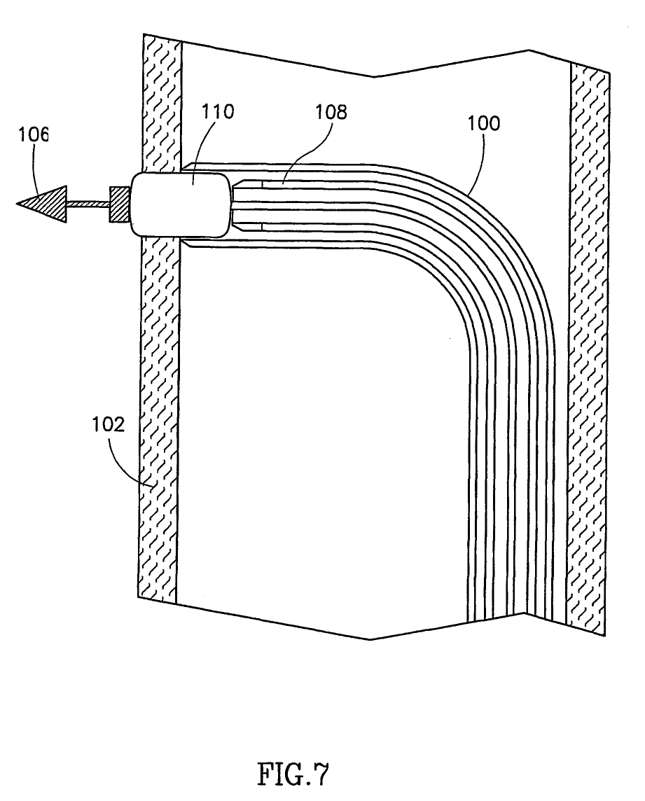

In FIG. 7, balloon 110 is inflated, expanding the opening in the aorta to be slight less, the same or even greater than the diameter of catheter 100. Optionally, the tip of outer tube 108 is not sharp, at least not on its inside edge. This may prevent the balloon from being damaged by the edge of tube 108.

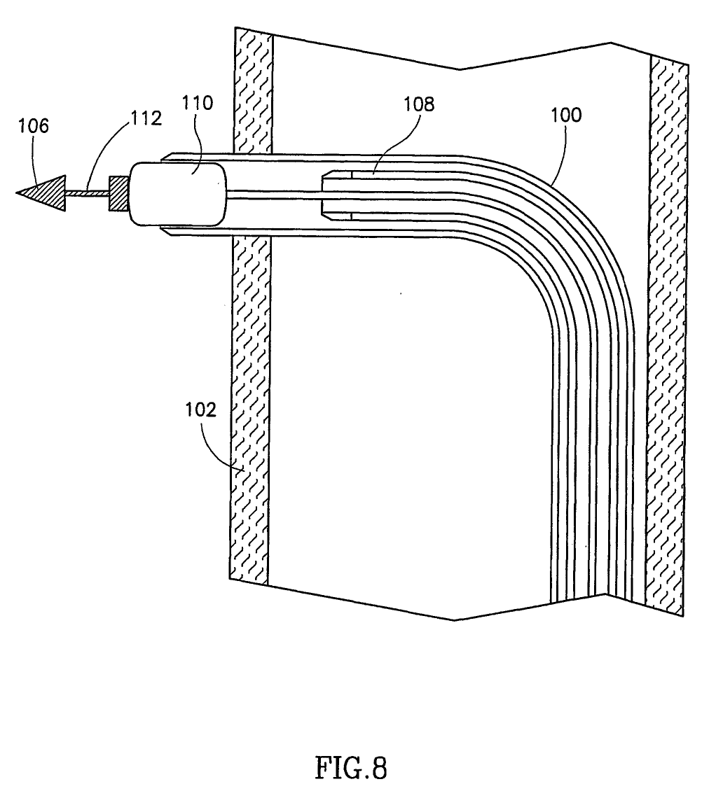

In FIG. 8, catheter 100 is advanced through the opening in aorta 102. Optionally, balloon 110 is inflated to engage catheter 100, so the two are advanced as one. Alternatively, catheter 100 is advanced over balloon 110.

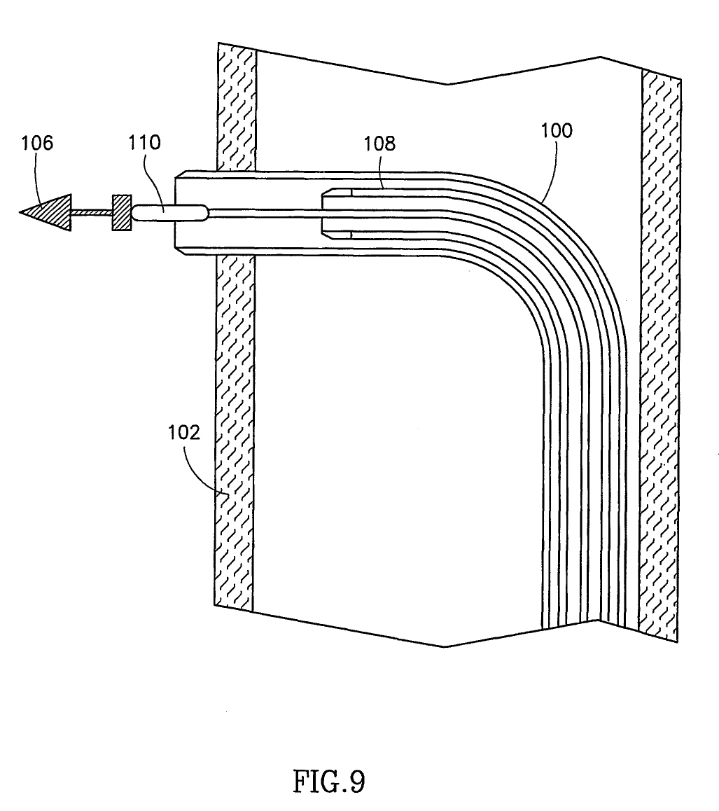

In FIG. 9, balloon 110 is deflated.

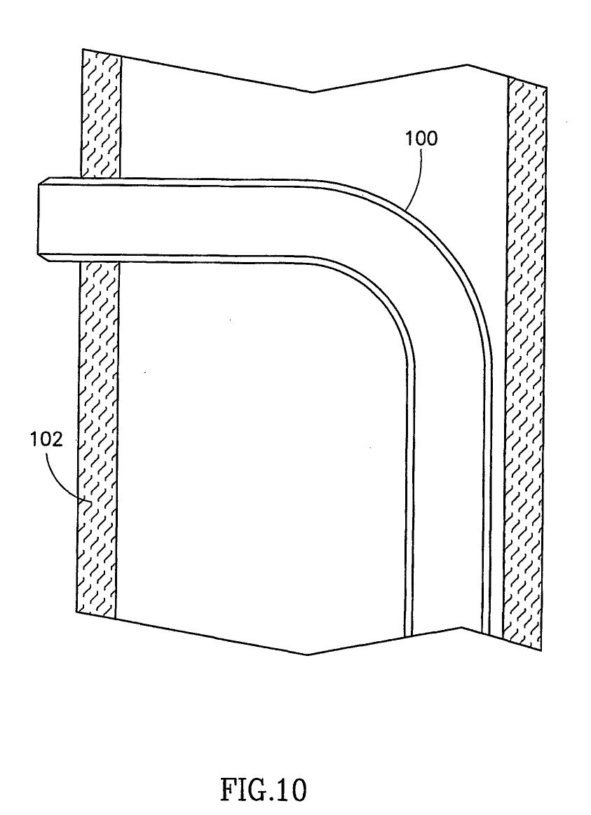

In FIG. 10, needle punch 106 is retracted with balloon 110, leaving catheter 100 transfixing the aorta

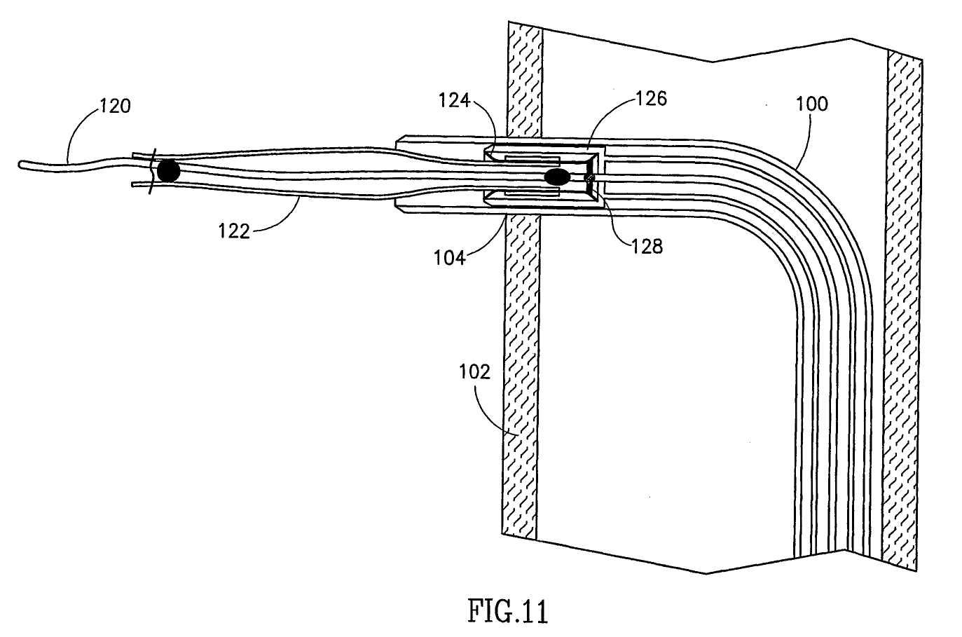

FIG. 11 shows a second stage of the anastomosis process in which a graft 122 (e.g., a vein, harvested artery or other graft type) is attached to aorta 102 at location 104. A guide wire 120 is optionally used for conveying graft 122 through catheter 100 and/or for navigation to the target vessel (not shown) various methods may be used for navigation, including, without limitation, X-ray fluoroscopy, ultrasound and MRI. Optionally, catheter 100 and/or other parts of the delivery system and/or portions thereof are made radio-opaque (or ultrasound reflecting) to assist in imaging the procedure.

Optionally, the contrast material that was previously injected outside the aorta is used as a reference for determining how far to advance the graft and/or connectors.

In an exemplary embodiment of the invention, graft 122 is provided attached to a connector 124. However, in other embodiments, the connector or the graft may be provided separate. In an exemplary embodiment of the invention, connector 124 is restrained in a delivery capsule 126, optionally using a holder 128.

In FIG. 11, capsule 126 is positioned so that the connector is inside the hole in aorta 102.

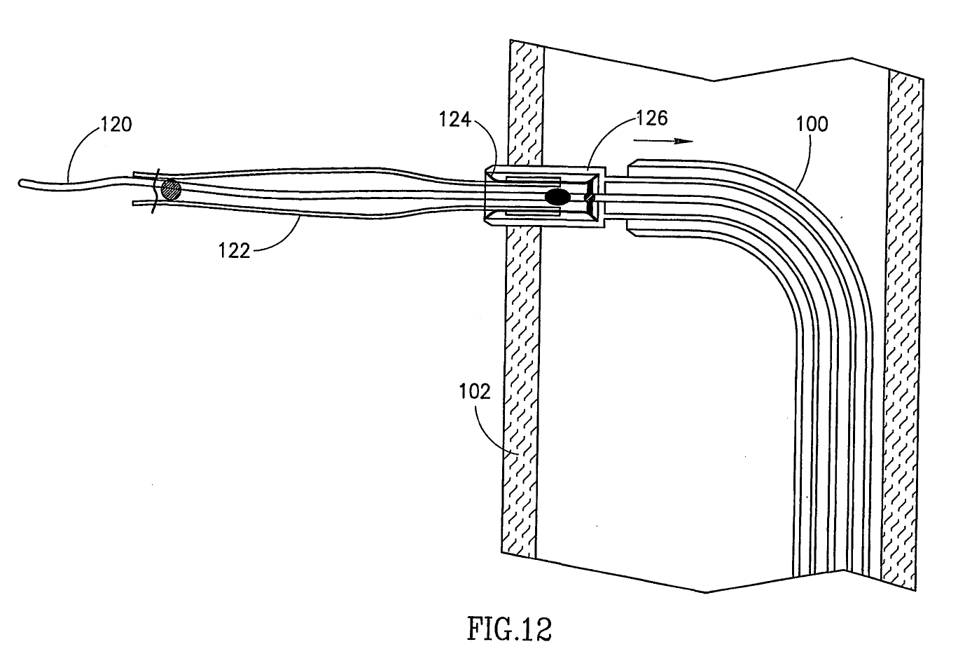

In FIG. 12, catheter 100 is retracted, leaving capsule 126 engaged by aorta 102. Possibly, this engagement is strong enough to prevent some or all leaks out of aorta 102.

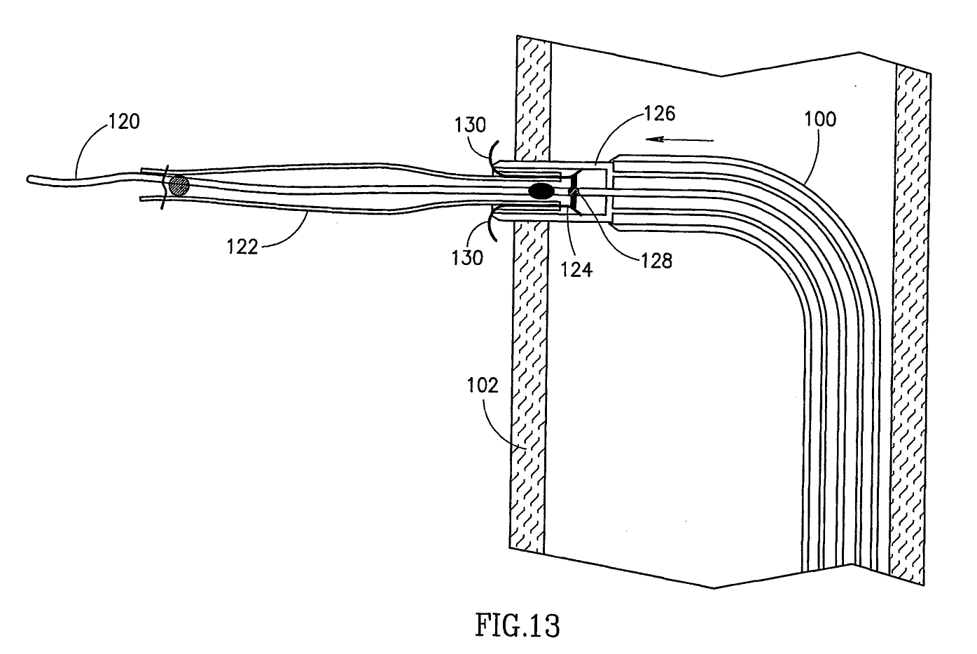

In FIG. 13, connector 124 is advanced relative to capsule 126, for example by advancing guide wire 120, which may be coupled to holder 128. A plurality of forward spikes 130 of connector 124 are thus freed from capsule 126 Optionally, capsule 126 is retracted alternatively or additionally to the advancement of connector 124.

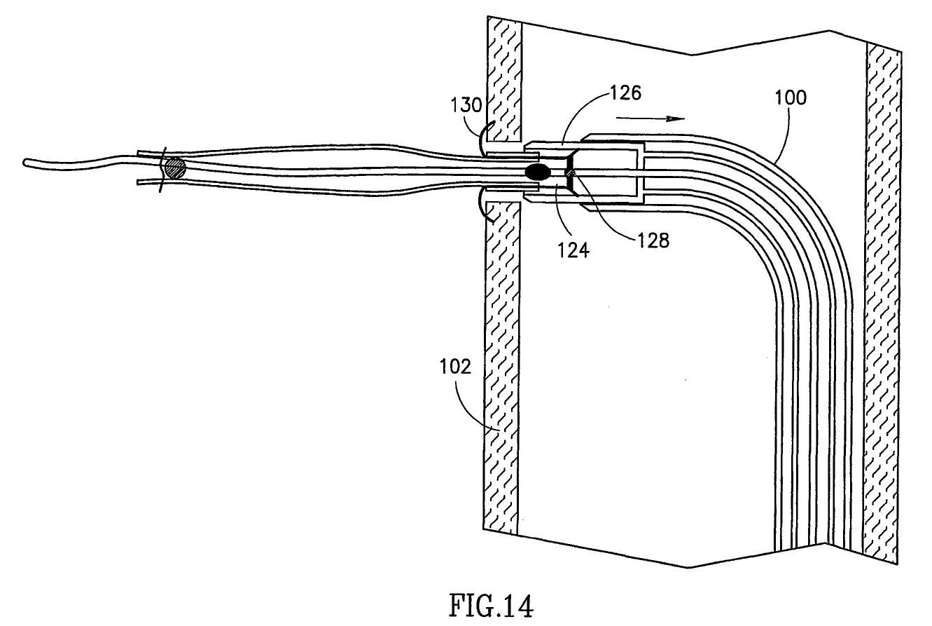

In FIG. 14, capsule 126 is retracted with connector 124, so that spikes 130 are pulled into the wall of aorta 102.

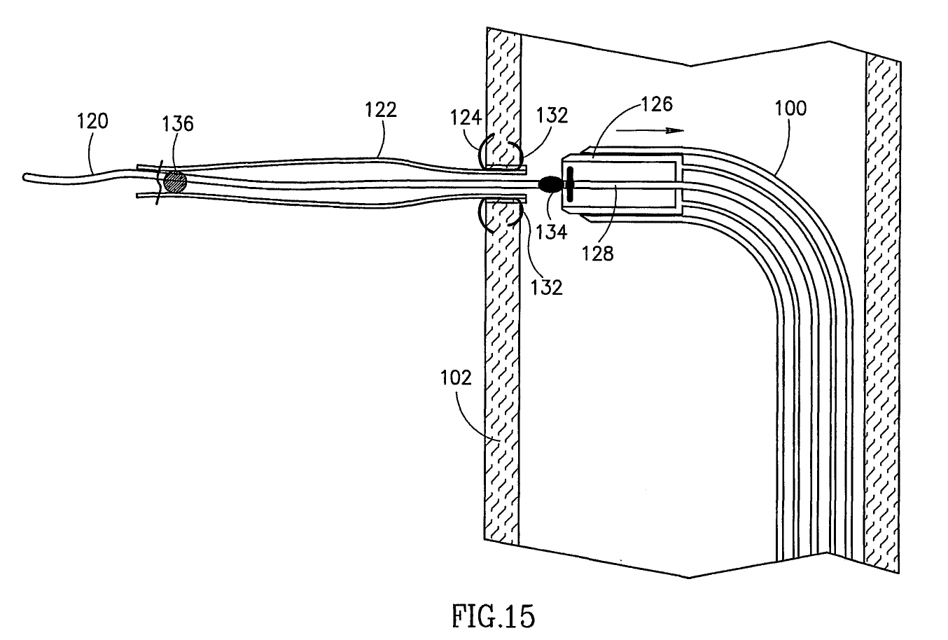

In FIG. 15, capsule 126 is further retracted, without connector 124, so that a plurality of backward spikes 132 of connector 124 are freed to engage aorta 102. The connection between aorta 102 and graft 122 is now complete. The other end of graft 122 may be connected to a target vessel in various manners, including by applying the same process in an opposite direction at the target vessel or through a mini-thoracic or keyhole opening.

Optionally, contrast material is injected into the graft and/or in the aorta near the graft. Such an injection allows to detect leaks from the connection or from the graft and/or to view the placement of all the connector legs relative to the aorta wall.

Two optional fat beads 134 and 136, that are fixed on guide wire 120, are shown. They may be used, for example, for radio-opaque imaging based techniques, such as fluoroscopy, to aid in verifying position and/or navigating. Alternatively or additionally, bead 134 may be used to apply force to holder 128 and/or keep it inside capsule 126. Holder 128 may, in different embodiments, be freely moving, coupled to guide wire 120, coupled to capsule 126 or riding on guidewire 120, with a ratchet mechanism that allow one direction of motion only. In an exemplary embodiment of the invention, holder 128 is a disk.

FIG. 16 illustrates a capsule 200 for guiding the delivery of an anastomosis connector, in accordance with an exemplary embodiment of the invention. This capsule may be used in place of capsule 126, in place of holder 128 and/or in addition to one or both of the parts, in different embodiments. As shown capsule 200 is formed of a slotted tube 202, in which the slots define a plurality of wings 204, which can swing out radially. Each wing has an inner rim 206 or other means for maintaining a tip of spike 130 in place. In an exemplary embodiment of the invention, capsule 200 releases spikes 130, when the wings exit (e.g., are pushed out) from capsule 126 and/or from aorta 102 (if there is no capsule).

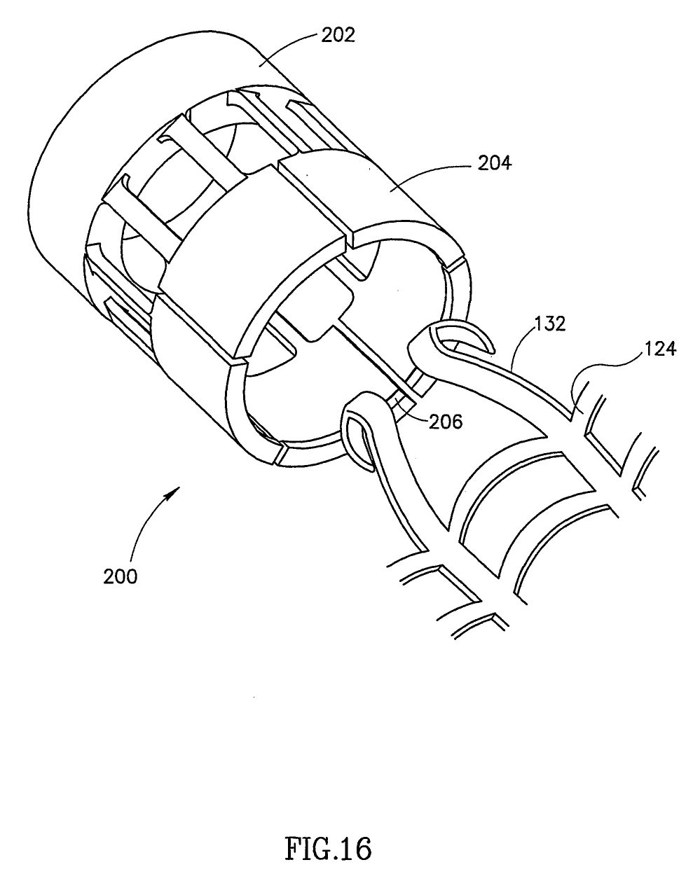

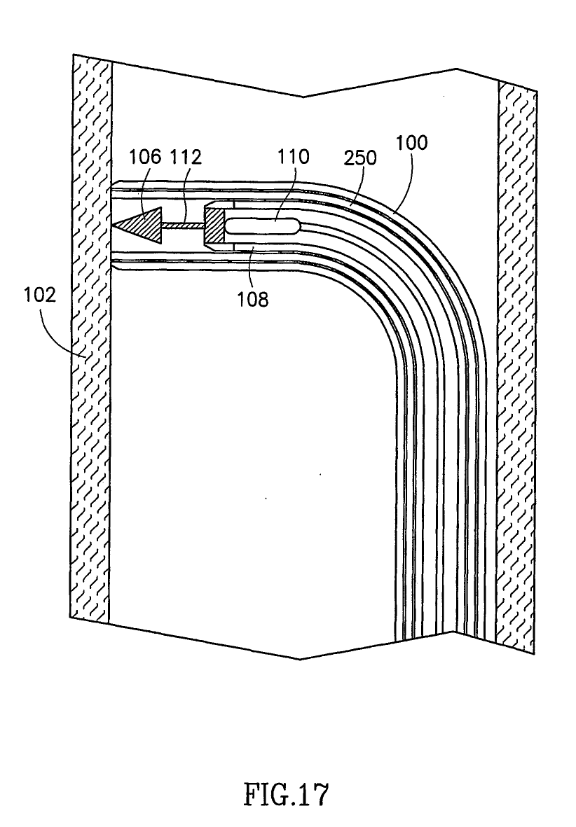

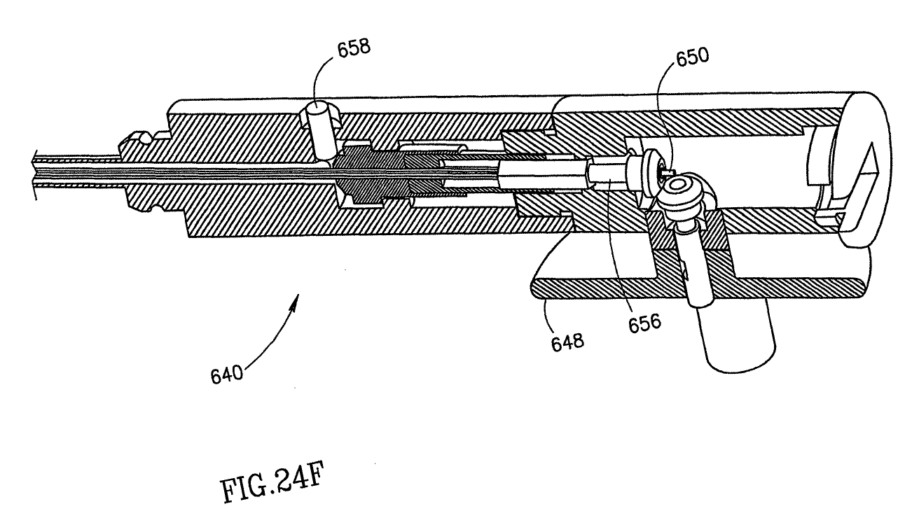

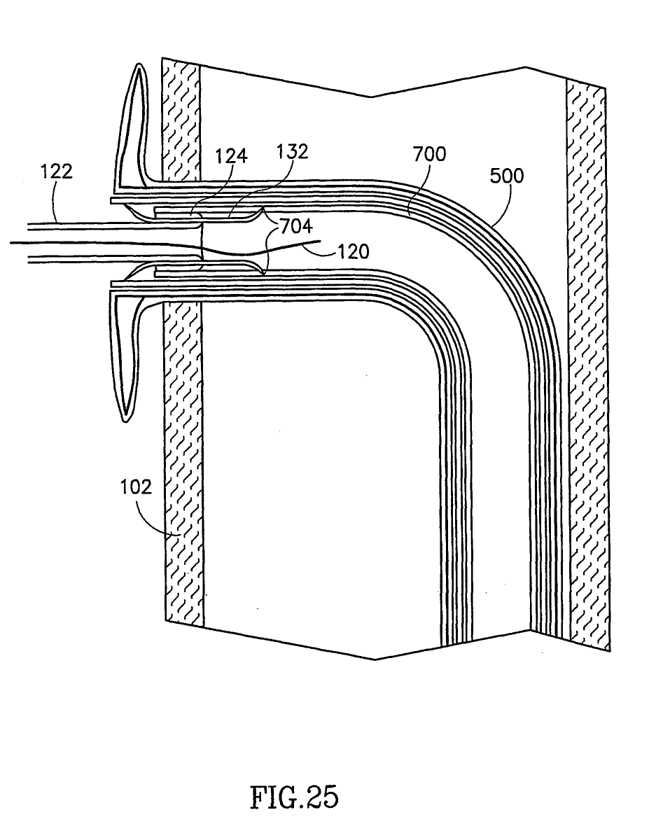

FIG. 17 illustrates an alternative delivery catheter system, including a separate protective sheath 250, within catheter sheath 100, in accordance with an exemplary embodiment of the invention. In this embodiment, a separate retractable/advancable sheath 250 is used to protect catheter 100 from punch 106. Optionally, sheath 250 is also used for guiding connector 124, as explained below in FIG. 25. The use of a balloon is optional, for example a thickening of the punch outer tube may replace the balloon, as described herein.

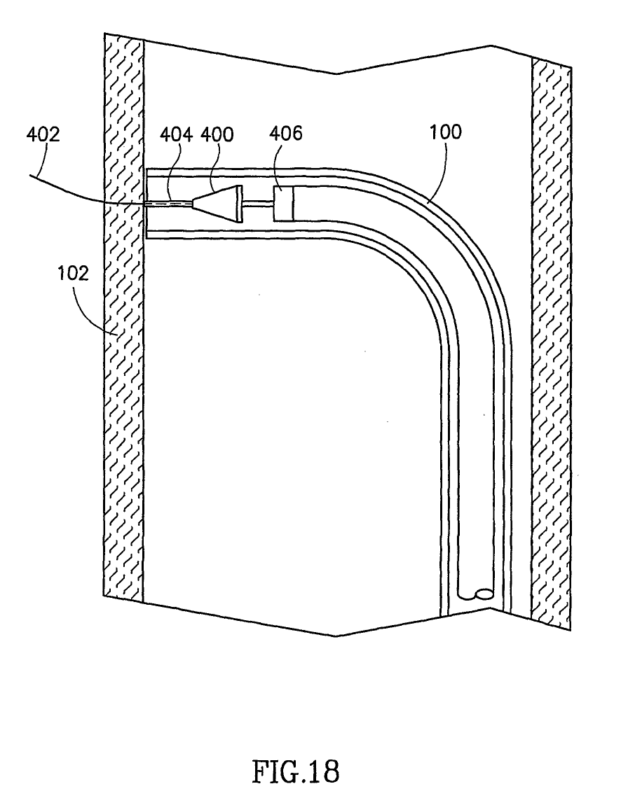

FIGS. 18-22 illustrate a guided punch, in accordance with an exemplary embodiment of the invention. The punch comprises a punch tip 400, which cooperates with a punch base 406, to remove a section of aorta 102.

In an exemplary embodiment of the invention, punch tip 400 is hollow, so that a sharp guide wire 402 can be extended there-through A pilot puncture in aorta 102 is made by wire 402. It should be noted that punch tip 400 does not then include a very sharp tip, so a protective sheath mechanism may be avoided, in some embodiments of the invention. The degree of extension of guide wire 402 may optionally be limited to the (expected) thickness of the aorta or less, in which case needle punch 400 is preferably brought against aorta 102 before guide wire 402 is extended. Alternatively, the extension is greater than the thickness, to ensure penetration of the aorta, for example, being between 3 mm and 10 mm. As noted above, contrast material may be injected through the sheath, to determine the aorta thickness.

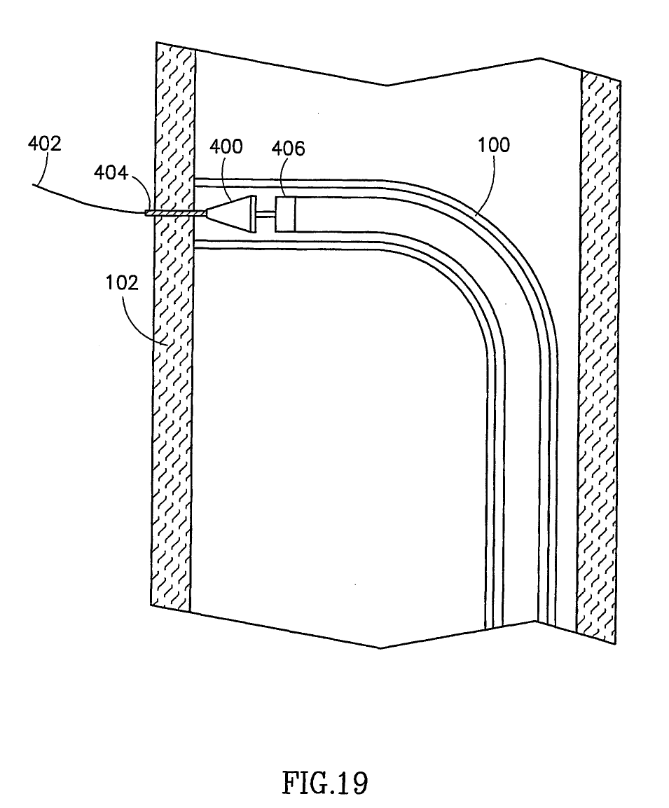

In FIG. 19, an optional tube 404 is advanced over the guide wire and through the aorta wall. This tube is thicker than the guide wire and may also serve to enclose the sharp tip of guide wire 402, to prevent inadvertent puncturing of nearby tissue. Alternatively, tube 404 may be an extension of punch tip 400. Once tube 404 is advanced, guide wire 402 is optionally retracted.

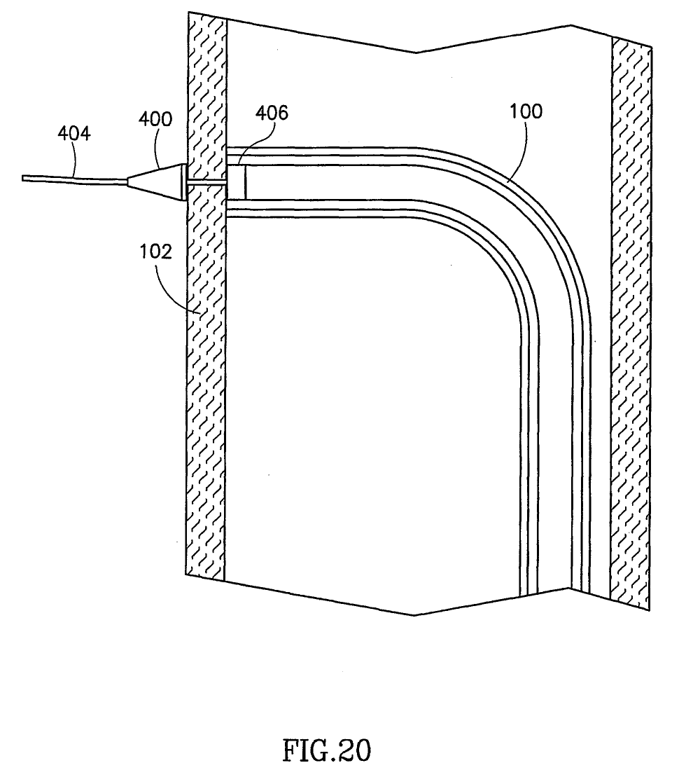

In FIG. 20, punch tip 400 is advanced over tube 404 (or guide wire 402 or just advanced), to penetrate the aortic wall, so the aortic wall is received between punch base 406 and punch tip 400.

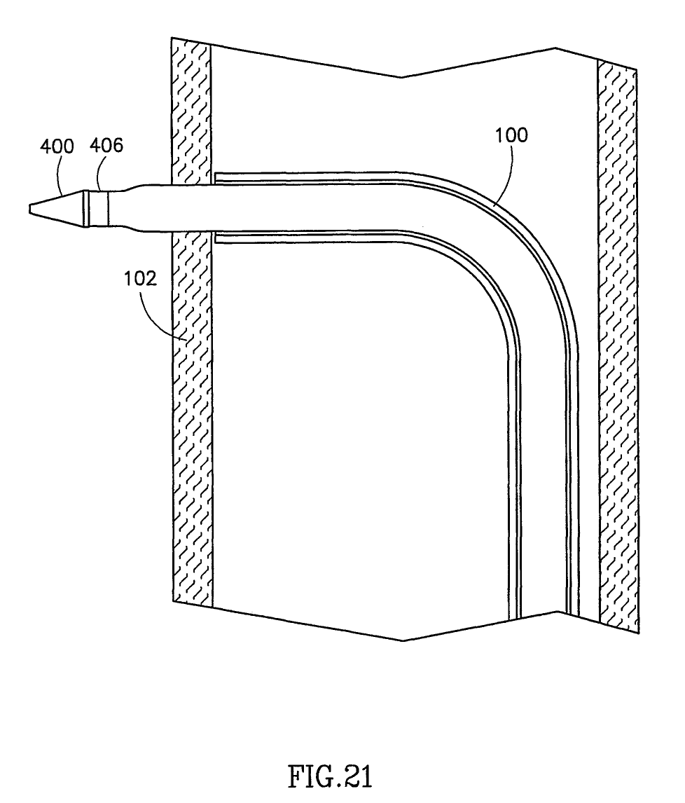

In FIG. 21, punch base 406 is advanced through the aortic wall, to punch out the received section. Optionally, base 406 (and optionally punch tip 400 as well) are then further advanced As shown, punch base 406 optionally thickens as it is advanced, so that its final outer diameter is near the inner (and outer) diameter of catheter 100 and the hole in the aortic wall is widened. Alternatively, a balloon may be used. Such a thickening method may be used as an alternative in FIGS. 1-15.

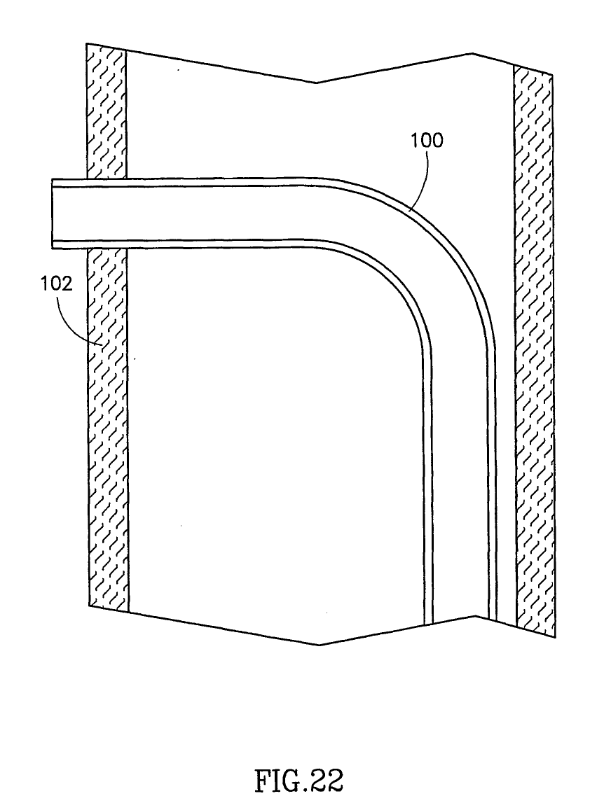

In FIG. 22, catheter 100 is advanced into the widened hole, as shown in FIG. 1, above.

A potential advantage of using a guide wire, is that if the needle punch is pushed to far ahead and then retracted out of the aorta wall, the guide wire can maintain the location of the hole formed by the punch, and prevent unnecessary damage of the aorta, caused by reinserting the punch at a second location.

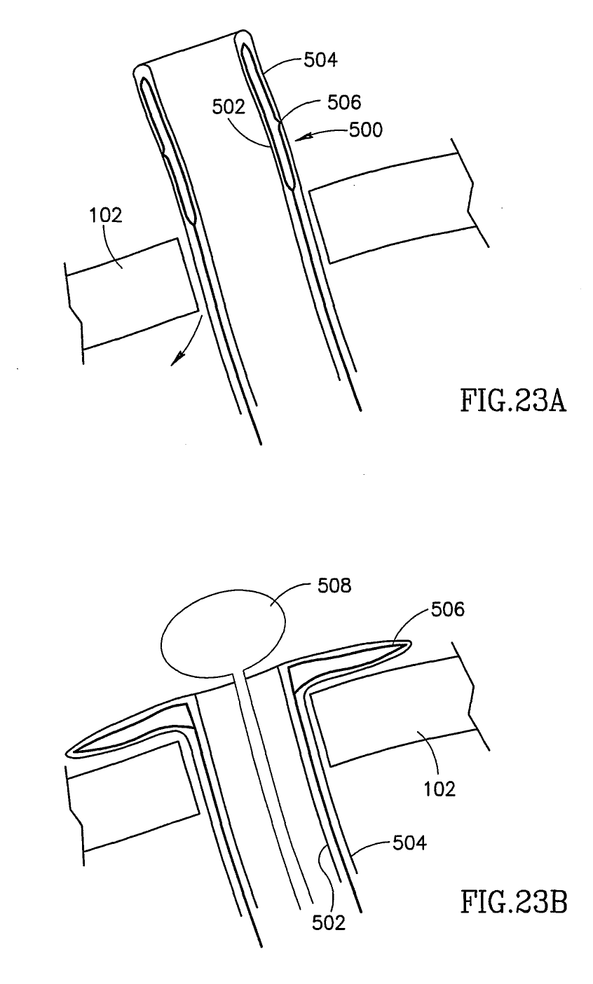

FIGS. 23A and 23B illustrate an anti-dislodgment mechanism for a catheter 500, in accordance with an exemplary embodiment of the invention. Catheter 500 comprises two layers, an inner layer 502 and an outer layer 504. In an exemplary embodiment of the invention, the separation into two layers is only at the tip of the catheter, with the outer layer 504 transforming into one or more axial cords away from the tip.

Optionally, catheter 500 is provided through guide catheter 100. In an exemplary embodiment of the invention, catheter 500 is conveyed through catheter 100, until its tip passes the opening in the aorta. Catheter 100 may then be retracted, so that the aorta engages catheter 500. Alternatively, catheter 500 maybe the only guiding catheter and replace catheter 100.

In FIG. 23A Catheter 500 is shown extending out of an aorta 102. However, in other uses, catheter 102 may be extending into a hollow body lumen, for example a blood vessel, a bladder or a digestive organ.

In FIG. 23B, inner layer 502 is retracted, while outer layer 504 is not, causing outer layer 505 to collapse, optionally about one or more pre-provided hinges 506, so that the outer diameter of the collapsed portion is significantly greater than the diameter of the opening. Optionally, a plurality of slots is formed in outer layer 504, to support such collapsing. Alternatively or additionally, to collapsing outside of aorta 102, the collapsing may take place within the aortic wall, albeit not with a same diameter increase.

A suitable positioning of hinges and slots (axially separated by a collar of unslotted material) will allow outer layer 504 to form to portions of increased diameter, one inside the aorta and one outside. Alternatively, only a collapsed portion external to the aorta is formed, for example by providing a collar of unslotted material at the tip of catheter 500.

Optionally a balloon 508 is temporarily inflated to assist and/or guide the collapsing, by actively widening the diameter of catheter 500.

Optionally, a thin membrane or balloon is provided over the tip of catheter 500, as part of the catheter, to prevent the slotted parts of outer layer 504 from inadvertently engaging any nearby tissue.

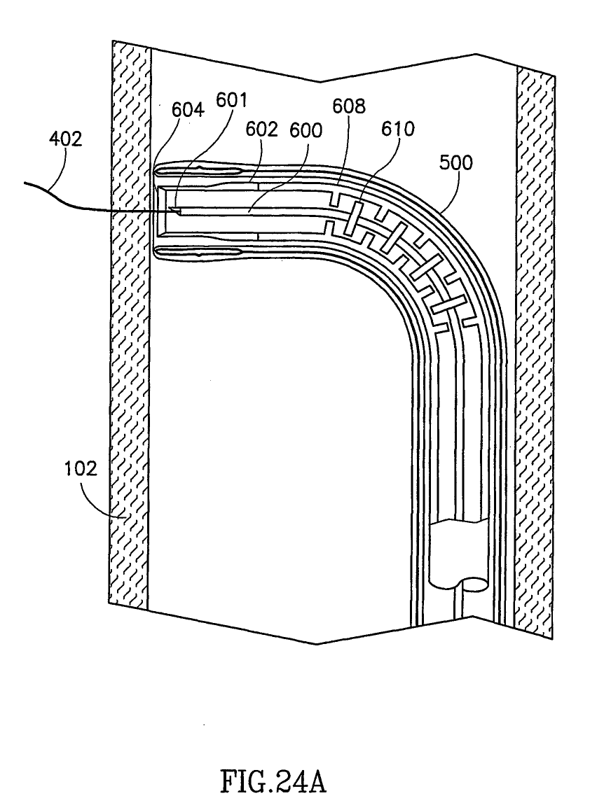

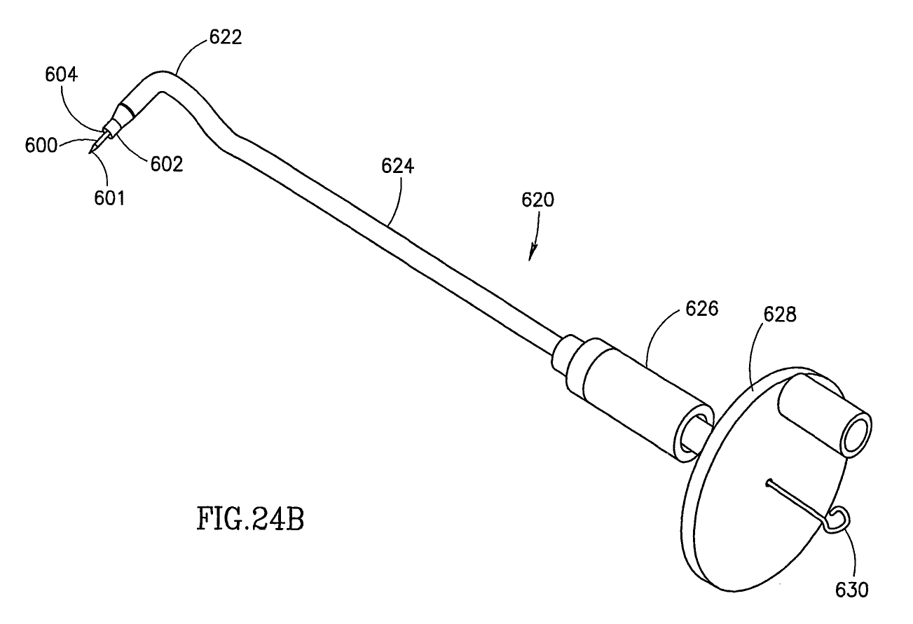

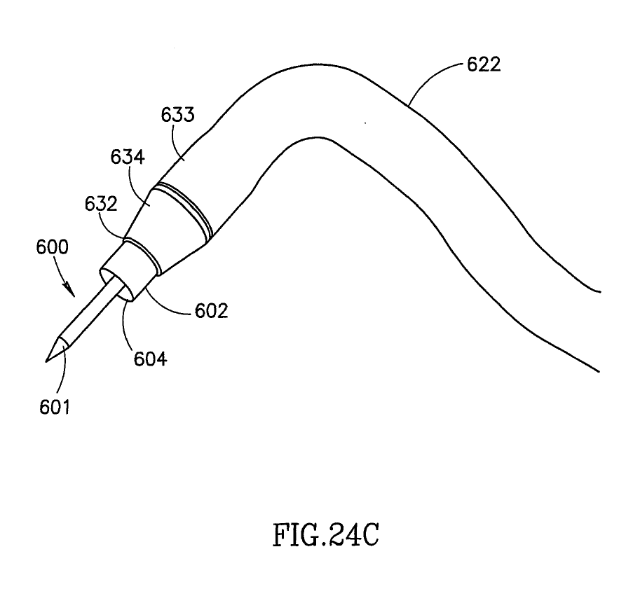

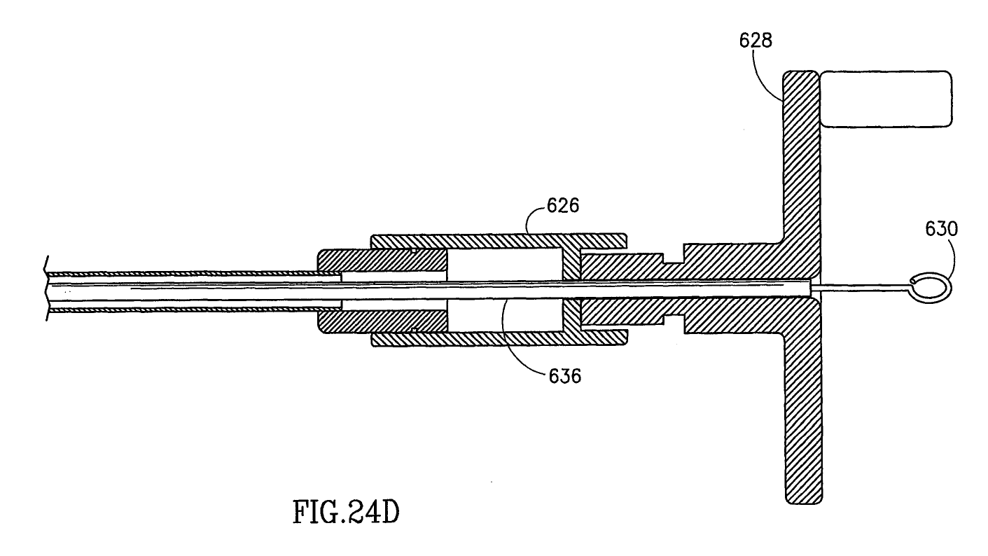

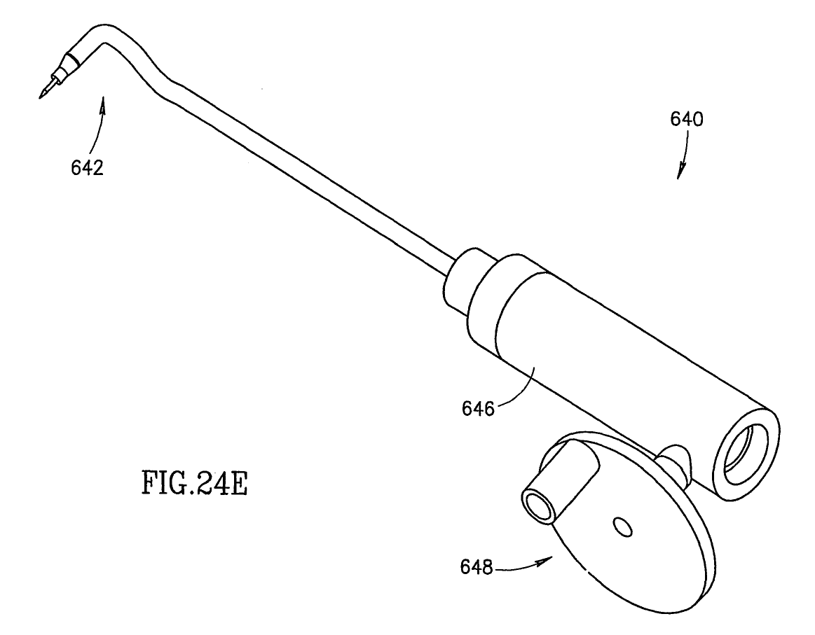

FIG. 24A illustrates a rotating and cutting out punch mechanism, in accordance with an exemplary embodiment of the invention. The mechanism is provided, for example, in catheter 500 and is used for cutting-out a section from an aorta 102.

In an exemplary embodiment of the invention, the mechanism comprises an inner pivot section 600 that is inserted into the aorta wall, anchoring in the wall or transfixing the wall. Optionally, pivot section 600 has a sharp tip 601. Alternatively or additionally, a sharp guide wire 402 (described above) is used to penetrate aorta 102. Optionally, tip 601 is barbed or inflatable or can be rotated to engage the aortic wall, for example using a threading (not shown). Thus, inadvertent retraction of tip 601 and/or motion of the punch, may be prevented. Optionally, as noted above, tip 601 may be replaced by a thin tube, which may be self flaring, for example as described below. An external cutting tube 602 has a sharp edge 604. Edge 604 may be smooth. Alternatively, it may be serrated, saw-tipped and/or may have a non-uniform diameter.

A plurality of threading sections 608 and 610 may couple tube 602 and pivot section 600. Alternatively, other methods may be used. In an exemplary embodiment of the invention, there is a significant empty space between tip 601 and edge 604. Tip 601 may be axially movable relative to edge 604, however, they may have a fixed relative position, for example tip 601 recessed or advanced relative to edge 604. In an exemplary embodiment of the invention, edge 604 advances towards tip 601, as it rotates. Such rotation may be used for various types of rotating punches, includes punches with a single cutting spike axially extending from edge 604

In use, tip 601 is inserted into aorta 102 and tube 602 is rotated around it. An outer tube is optionally advanced into the hole thus formed Tip 601 and/or tube 602 are then retracted.

FIGS. 24B-24D show an exemplary rotating punch 620, in accordance with an exemplary embodiment of the invention. Punch 620 comprises a head 622 (one exemplary embodiment of which is described in general in FIG. 24A), an elongate shaft 624, adapted for passing through a catheter or an endoscope, a handle 626 and a rotatable cam 628. In an exemplary embodiment of the invention, cam 628 is coupled to tube 602. Optionally, tip 601 is attached to an external grip 630 for selectively advancing and/or retracting tip 601.

FIG. 24C is a close-up of head 622, showing an optional (non-rotating or freely rotating) outer sheath 633, having a narrowing cone 634 terminating at a lip 632. In an exemplary embodiment of the invention, cone 634 is used to advance sheath 633 into an opening created by cutting edge 604. Optionally, tube 602 and/or cone 632 are retracted, allowing the use of sheath 633 as a delivery guide. Alternatively, cone 634 is used to widen the punched hole, to assist in advancing the outer sheath (e.g., catheter or endoscope) into the punched hole.

FIG. 24D is a cross-sectional view of handle 626, showing a hollow inner shaft 636 through which a retractable tip 630 is advanced.

FIGS. 24E-24F show an alternative rotating punch 640, in accordance with an exemplary embodiment of the invention. A rotating cam 648 is set on a side of a body 646 of punch 640. A head 642 can be the same head 622 of FIG. 24B.

FIG. 24F is a view of the working mechanism of punch 640, showing the rotation of a shaft 656, while allowing an inner guide wire 650 to remain stationary and/or be moved axially. An optional safety pin 658 is also shown, for preventing inadvertent rotation of shaft 656.

FIG. 25 illustrates a device delivery guide 700, as an alternative to the capsule shown in FIG. 16, in accordance with an exemplary embodiments of the invention. In guide 700, the tips of backward spikes 132 of connector 124 are engaged in a plurality of holes 704, in a tubular element 700.

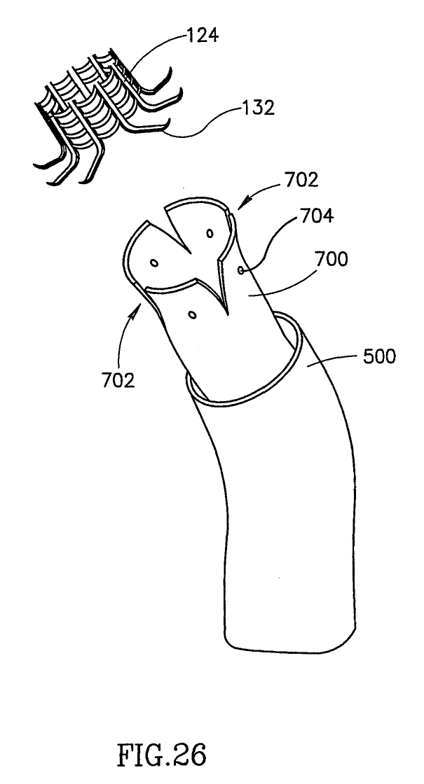

FIG. 26 is an exploded view of the guide 700, showing that a plurality of wings 702 is formed at the end of guide 700, such that when they flare out, holes 704 release the tips of spikes 132.

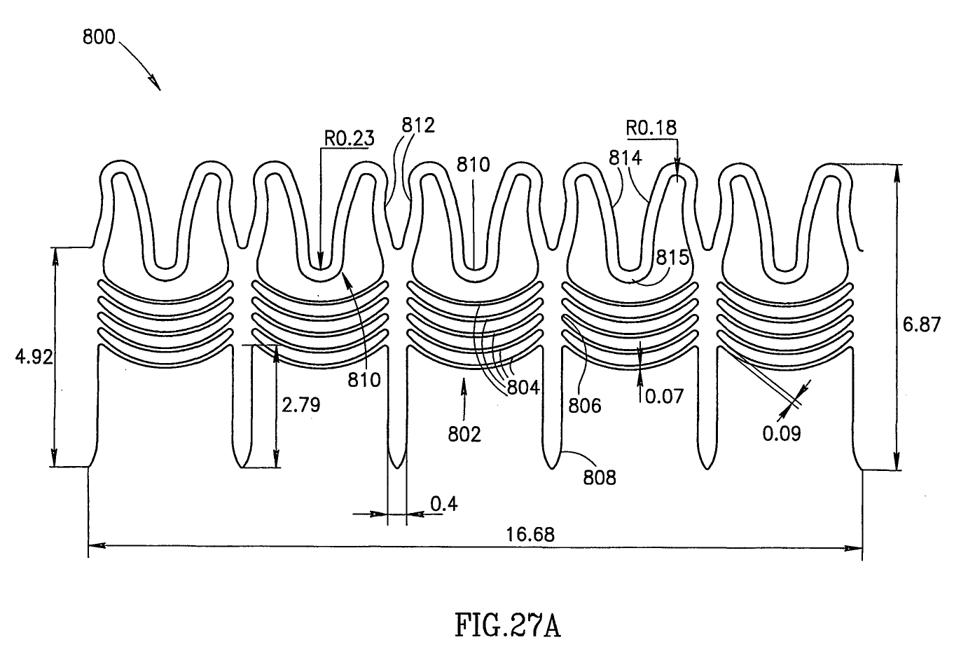

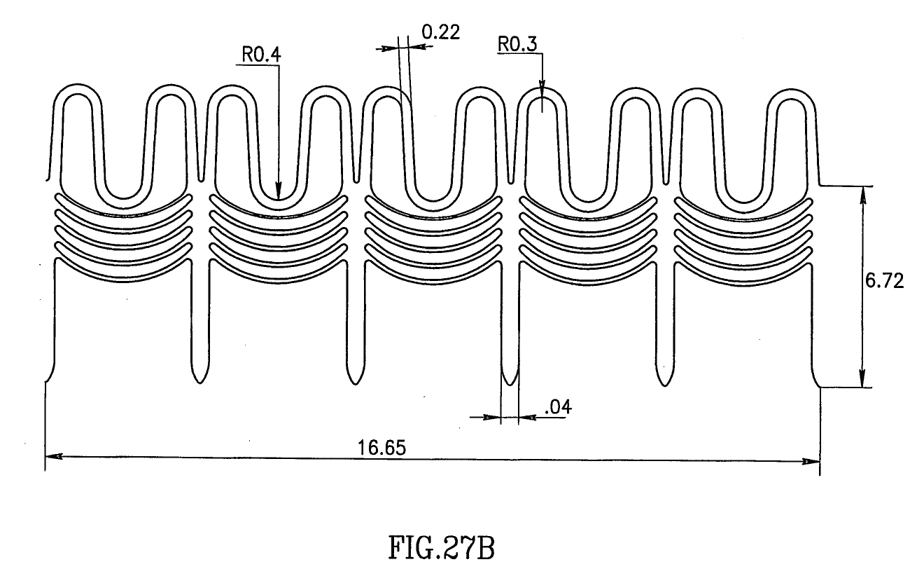

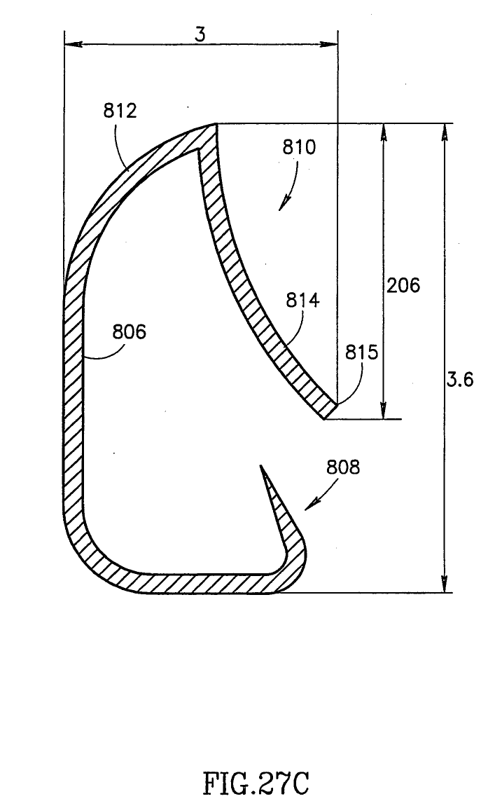

FIGS. 27A-27C illustrate two exemplary anastomosis connectors, in accordance with an exemplary embodiment of the invention. FIG. 27A shows a connector 800, in plan view having a body 802 comprised of a plurality of arcs 804 that interconnect adjacent spikes segments 806. Spike segments 806 extend in one direction (the backwards direction), away from body 802, to form a plurality of spikes 808. In the opposite direction, spike segments 806 extend to form bases for a plurality of non-penetrating spikes 810. In an exemplary embodiment of the invention, each of spike segments 806 splits into two bases 812, however, this is not required. In an exemplary embodiment of the invention, spikes 810 are formed of two arms 814 that meet at a spike tip 815 and are attached at their other ends to spike bases 812, of adjacent spike segments 806. In an exemplary embodiment of the invention, arms 814 and bases 812 define an undulating curve. The exemplary dimensions shown are in mm.

FIG. 27B shows an alternative, embodiment, in which the form of the curve is different. Possibly, the form of FIG. 27A allows greater force to be applied by the twisted joints. Alternatively, the joints may be replaced by straight torsion bars. Optionally, the torsion bars are made thinner or weaker than the surrounding connector, to ensure that they twist. Optionally, the form of the curve is adapted to match a bending pattern of the undulating curve, as shown in FIG. 27C.

FIG. 27C shows a side cross-sectional view of a single spike segment 806 of connector 800, showing an exemplary bend configuration of the spikes. Optionally, the sharp bends are achieved by twisting the spikes. In an exemplary embodiment of the invention, the spikes are pre-bent and connector 800 is elastic, super-elastic or shape memory, so that it attempts to return to the geometry shown in FIG. 27C, when delivered. Alternatively, connector 800 is a plastically deformed connector.

As shown, in an exemplary embodiment of the invention, spike 808 is a penetrating spike that is bent twice 90°. In an exemplary embodiment of the invention, the bending is performed by twisting of the spike, e.g., arms 814 or bases 812. Spike 810 is a non-penetrating spike mounted on bases 812 (one shown). Base 812 is curved or bent away from segment 806. Then, base 812 bends (or is twisted) at the point of attachment to arm 814. Arm 814 is optionally curved so that tip 815 when contacting a vessel wall will tend to bend away from the wall, rather than attempt to penetrate it.

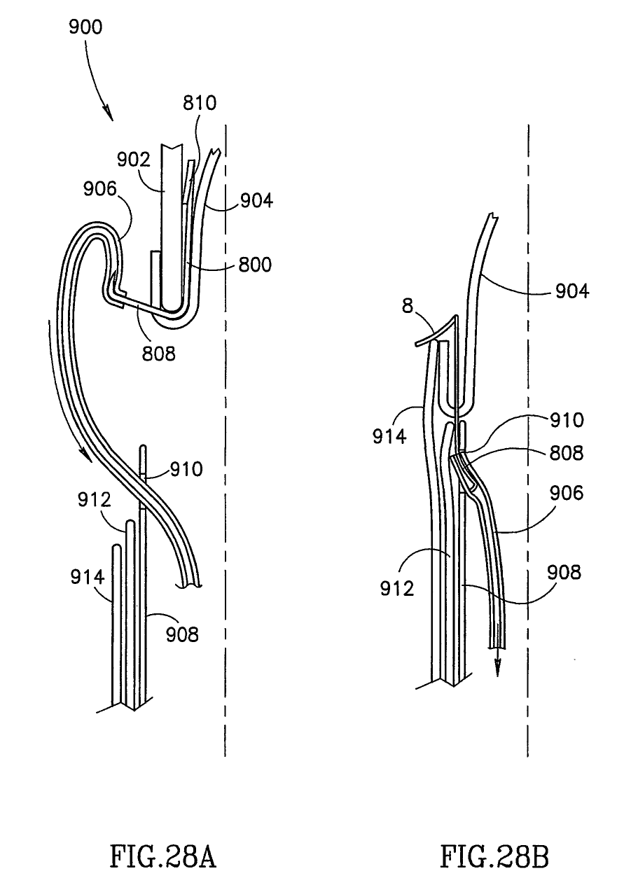

FIGS. 28A-28B illustrate a method of mounting a connector, such as connector 800, into a delivery system 900, in accordance with an exemplary embodiment of the invention. FIG. 28A shows connector 800 mounted in a loading tube 902. A graft 904 is everted over connector 800 and transfixed by spikes 808. Spikes 810 are held between the graft and loading tube 902.

A thin, flexible tube 906 is mounted on each spike 808 and passed through a slot 910 of an inner window tube 908 of delivery system 900. An intermediate, locking tube 912 is optionally provided between window tube 908 and an outer tube 914.

FIG. 28B shows the effect of pulling back on all the flexible tubes 906 substantially simultaneously. Graft 904 is pulled out of loading tube 902. Spikes 810 (released from tube 902) are optionally allowed to open and engage the outer lip of tube 914. Spikes 808 are pulled into slots 810. In an exemplary embodiment of the invention, locking tube 912 is advanced, locking connector 800 between locking tube 912 and window tube 908. Further retraction of tubes 906 will thus only cause the removal of tubes 906 from spikes 808 and not further retraction of connector 800. Connector 800 is then optionally released, by retracting locking tube 912.

It should be noted that locking connector 800 and/or the use of holding slot 910 potentially allow connector 800 to be selectively pulled or pushed within outer tube 914.

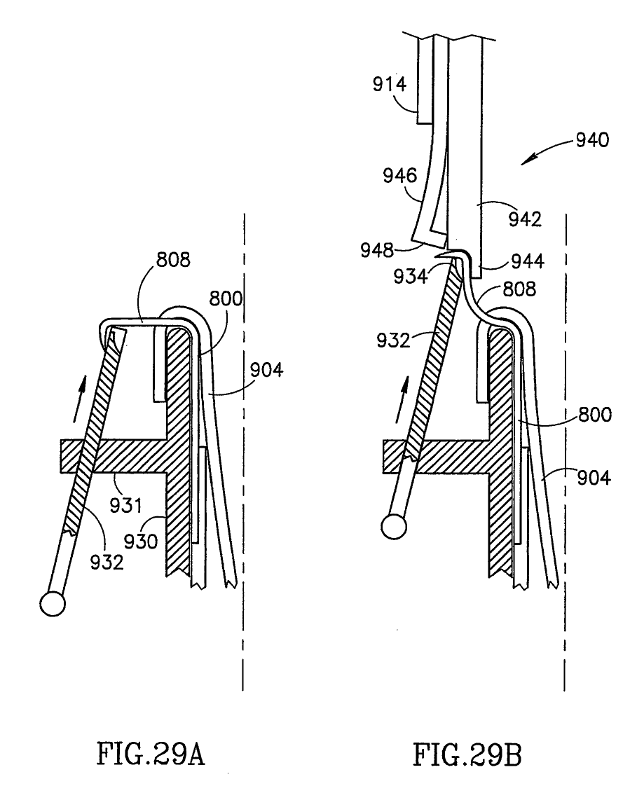

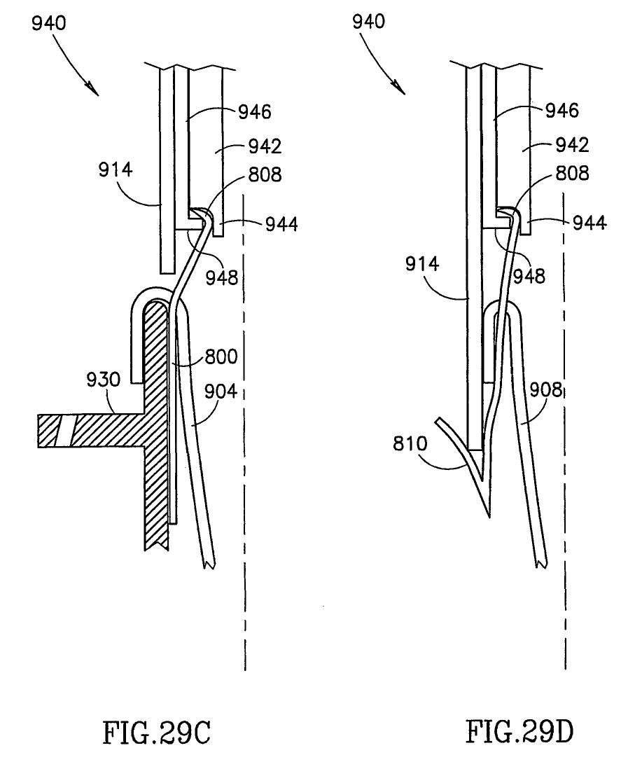

FIGS. 29A-29D show a method of mounting connector 800, in accordance with an alternative exemplary embodiment of the invention. A graft loader 930 restrains a connector 800, which transfixes an everted graft 902. Unlike holder 902 of FIG. 28A, holder 930 includes one or more pins 932, for folding pikes 808 back into a delivery system 940 (FIG. 29B). In an exemplary embodiment of the invention, holder 930 includes a ring 931 defining a plurality of through channels for a plurality of pins 932, one for each spike 808. Alternatively, a single pin is used for all spikes, in series.

In FIG. 29B, a forward tip 934 of pin 932 advances and bends spike 808 back. In an exemplary embodiment of the invention, delivery system 940 comprises outer tube 914 and an inner tube 942, having an extending inner lip 944. Tip 934 pushes spike 808 against inner lip 944. A plurality of spike holders 946, having inwards extending fingers 948 are provided to engage the tip of spikes 808. Optionally, spikes holders 946 comprise sections of a single slotted tube. As shown, fingers 948 are proximal to the end of tube 942, for example, by advancing tube 942 further than spike holders 946, out of outer tube 914.

In FIG. 29C, holders 946 are advanced, so that the tip of spike 808 is held between finger 948, inner lip 944 and the front lip of tube 942. Both holders 946 and tube 942 are optionally retracted, so that pulling hard on connector 800 will not inadvertently dislodge spikes 808.

In FIG. 29D, delivery system 940 is retracted relative to graft holder 930, so that connector 800 and graft 902 are pulled off of holder 930. Optionally, spikes 810 open and engage tube 914.

In an exemplary embodiment of the invention, the graft holder uses a graft conveying element in the shape of a flexible element with a retractable pin at its end. Such an element is described, for example in PCT/IL01/00069, the disclosure of which is incorporated herein by reference.

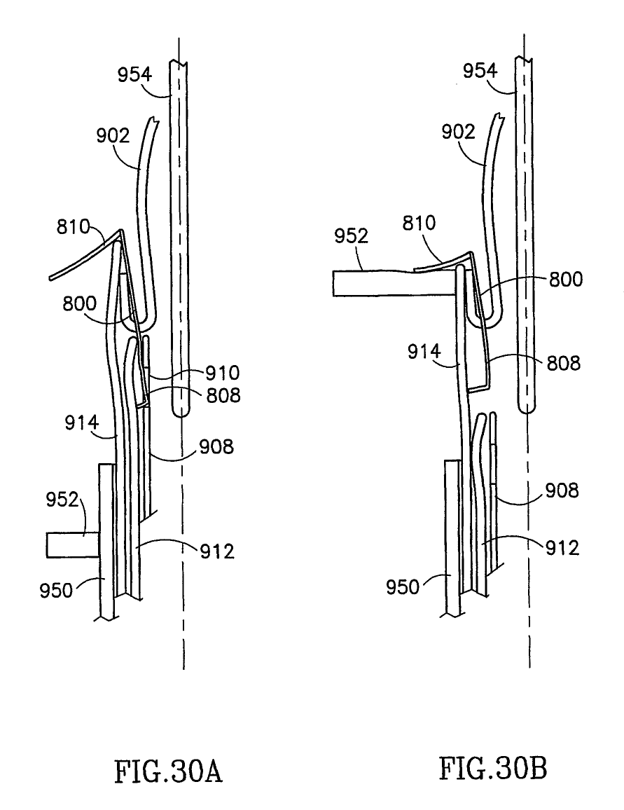

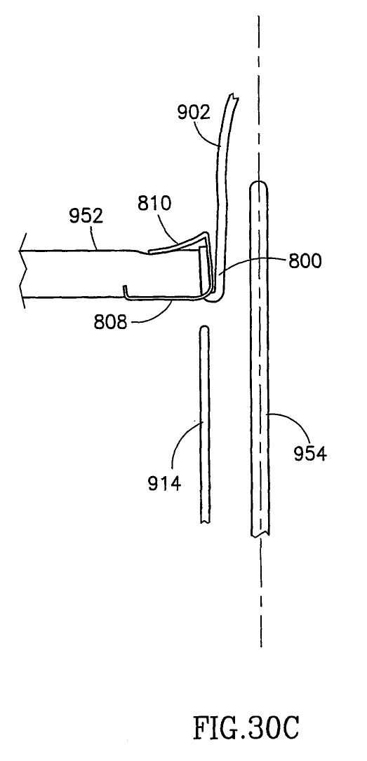

FIGS. 30A-30C show details of the process of attaching connector 800 to an aorta 952, in accordance with an exemplary embodiment of the invention, which does not necessarily require a capsule. In FIG. 30A, a hole has been punched in aorta 952 and a guide sheath 950 inserted in the hole, optionally plugging it. A delivery system including outer tube 914 and a graft 902 is advanced through sheath 950 and past the wall of aorta 952, optionally along a guide wire 954.

In FIG. 30B, guide sheath 950 is retracted out of the opening in the aorta, so that the wall of aorta 952 engages outer tube 914 instead. In addition, outer tube 914 is retracted sufficiently to allow non-penetrating spikes 810 to contact aorta 952. In other embodiments, penetrating spikes are used. One potential advantage of non-penetrating spikes is that there is less danger of inadvertently damaging tissue or catching on tissue outside the aorta by the spikes.

Connector 800 is unlocked (in this implementation) by retracting first locking tube 912 and then window tube 908. The extended spikes 810 prevent retraction of connector 800.

In FIG. 30C, outer tube 914 is retracted, freeing spikes 808 to bend and engage aorta 952 opposite spikes 808, completing the anastomotic connection of graft 902 to aorta 952.

In an exemplary embodiment of the invention, the above or other methods of performing a bypass are used to connect a venous system to an arterial system, such that the venous system serves as a conduit for oxygenated blood.

In an exemplary embodiment of the invention, a graft is connected between the aorta, a mammary artery or other artery to the coronary sinus and/or to one or more of the coronary veins.

In an embodiment where the connection is to the coronary sinus, the connection between the coronary sinus and the vena cava is sealed, for example, using a suture, an internal suture, a clogging device or any other means of sealing blood vessels known in the art. Optionally, at least one of the coronary veins is disconnected from the coronary sinus and connected to the venous system, to provide some measure of venous drainage.

In an embodiment where the connection from the aorta is to a coronary vein, the connection of the vein to the coronary sinus is severed.

The access for performing the bypass procedures may be of any type known in the art, for example, transvascular, thoracic or using open surgery.

It will be appreciated that the above described methods of providing a tools and bypassing may be varied in many ways, including, changing the order of acts, which acts are performed more often and which less often, the arrangement of the tools, the type and order of tools used and/or the particular timing sequences used. Further, the location of various elements may be switched, without exceeding the sprit of the disclosure. In addition, a multiplicity of various features, both of methods and of devices have been described. It should be appreciated that different features may be combined in different ways. In particular, not all the features shown above in a particular embodiment are necessary in every similar exemplary embodiment of the invention. Further, combinations of features from different embodiments into a single embodiment or a single feature are also considered to be within the scope of some exemplary embodiments of the invention. In addition, some of the features of the invention described herein may be adapted for use with prior art devices, in accordance with other exemplary embodiments of the invention. The particular geometric forms and measurements used to illustrate the invention should not be considered limiting the invention in its broadest aspect to only those forms. Although some limitations are described only as method or apparatus limitations, the scope of the invention also includes apparatus designed to carry out the methods and methods of using the apparatus.

Also within the scope of the invention are surgical kits, for example, kits that include sets of delivery systems and anastomotic connectors. Optionally, such kits also include instructions for use. Measurements are provided to serve only as exemplary measurements for particular cases, the exact measurements applied will vary depending on the application. When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. An anastomosis delivery system for delivering a connector having at least one backwards spike having a bent tip, comprising:
a hollow guide sheath; and
a hollow, axially slotted section, fitting within said sheath, said section having a flared configuration and an unflared configuration and wherein said axially slotted section is adapted to contain at least a part of said connector and to limit axial motion of said connector when said section is in its unflared configuration.

2. A system according to claim 1, wherein axially moving said section selectively advances said spike.

3. A system according to claim 1, wherein axially moving said section selectively retracts said spike.

4. A system according to claim 1, wherein said slotted section maintains said bent tip in a bent configuration.

5. A system according to claim 1, wherein said slotted section includes at least one receptacle for engaging said bent tip.

6. A system according to claim 5, wherein said receptacle comprises an inner lip of said section, adapted for catching said tip.

7. A system according to claim 5, wherein said receptacle comprises a hole in said section, for engaging said tip.

8. A system according to claim 1, wherein said section comprises a second, inner tube and wherein said inner tube and said slotted section define between them a receptacle for a bent section of at least one bent spike of connector.

9. A system according to claim 8, wherein said receptacle is a space between tips of said slotted section and said inner tube.

10. A system according to claim 7, wherein said receptacle is an opening in said inner tube.

11. A system according to claim 7, wherein said slotted section and said inner tube grip between them a part of said connector.

12. A system according to any of claims 1-11 wherein said slotted section comprises a capsule closed at one end.

13. An anastomosis delivery system for delivering a connector having at least one backwards spike having a bent tip, comprising:
a hollow guide sheath;
an apertured inner tube fitting within said sheath; and
a plurality of spike locking elements disposed between said guide sheath and said apertured inner tube, wherein said spike locking elements, when extended, are adapted to grip a part of said anastomosis connector between said inner tube and said locking elements and wherein said apertures are each adapted to receive a said bent tip of said anastomosis connector.

14. An anastomosis delivery system for delivering a connector having at least one backwards spike having a bent tip, comprising:
a hollow guide sheath;
a cylindrical capsule having one open end an one closed end; and
an anastomosis connector held in said capsule.

15. A system according to claim 14, comprising a stopper arranged between a plurality of said backwards spikes and urging said spikes towards said capsule

16. A method of mounting an anastomosis connector having a plurality of bent backwards spikes including bent tips, into a delivery tube, comprising:
bending back said spikes to point backwards along an axial direction of said connector, away from a graft mounted on said connector;
maintaining said tips in a bent configuration; and
inserting said spikes into a receptacle of said delivery tube, which receptacle maintains said tips in a bent configuration.

17. A method according to claim 16, wherein bending back comprises:
mounting a thin flexible tube on each of said spikes;
threading said tube through a plurality of tip holding apertures in said receptacle; and
retracting said tubes to bend said spikes and pull them into said receptacle.

18. A method according to claim 17, comprising:
locking said connector in place; and
retracting said tubes to remove them from said spikes.

19. A method according to claim 16, wherein bending back comprises:
pushing back each spike, using a jig, into said receptacle; and
locking said spike tip in said receptacle.

20. A guided punch, comprising:
a sharp, extendible guide wire; and
a hollow punch mechanism adapted to ride on the guide wire, wherein said guide wire is adapted to extend from said punch.

21. A punch according to claim 20, wherein said guide wire has a limited extension distance of less than 3 cm.

22. A punch according to claim 21, wherein said distance is shorter than 1 cm.

23. A punch according to claim 21, wherein said distance is greater than 0.3 cm.

24. A punch according to any of claims 20-23, wherein said punch comprises a hollow tube adapted to fit between said punch mechanism and said guide wire.

25. A punch according to any of claims 20-23, wherein said punch is a rotating punch.

26. A punch according to any of claims 20-23, wherein said punch is an axially moving punch.

27. A punch according to any of claims 20-23, wherein said punch is adapted for injection of contrast material inside of said hollow of said punch mechanism.

28. A rotating punch, comprising:
a sharp, central guide wire; and
a rotating outer tube having a vascular cutting edge defined by a lip of said tube.

29. A punch according to claim 28, wherein said outer tube advances as it is rotated.

30. A punch according to claim 29, wherein said advancing is limited to less than 3 cm.

31. A punch according to claim 29, wherein said advancing is limited to less than 1 cm.

32. A punch according to claim 29, wherein said punch is adapted for a particular target vessel, by matching said advancing limitation to the target vessel.

33. A punch according to claim 28, wherein said cutting edge is smooth.

34. A punch according to claim 28, wherein said cutting edge is serrated.

35. A punch according to claim 28, wherein said guide wire is smooth.

36. A punch according to claim 28, wherein said guide wire is adapted to engage vascular tissue it is inserted into.

37. A punch according to claim 28, comprising a hollow tube adapted to be brought over said guide wire and within said rotating outer tube.

38. A punch according to claim 37, wherein said punch is adapted for injection of contrast material inside of said hollow tube.

39. A punch according to any of claims 28-37, wherein said punch is adapted for injection of contrast material between said spike and said outer tube.

40. A punch according to any of claims 28-37, wherein said outer tube is bent at a right angle, such that positioning perpendicular to a vessel wall is assisted.

41. A punch according to any of claims 28-37, wherein said outer tube has an increasing outer diameter, away from said cutting edge.

42. A punch according to any of claims 28-37, comprising a balloon distal from said cutting edge, said balloon, when inflated, having an outer diameter slightly greater than a diameter of said outer tube and about the inner diameter of a sheath associated with said punch.

43. An advancing rotating punch, comprising:
a sharp, central guide wire; and
a rotating outer tube adapted to cut a target vessel which advances relative to said wire when it rotates.

44. A catheter system, comprising:
an outside sheath having an inner volume;
a first contrast injection port communicating with the inner volume of said sheath;
at least one inner mechanism conveyed by said sheath and having an inner volume; and
a second contrast injection port communicating with the inner volume of said inner mechanism.

45. A system according to claim 44, wherein said at least one inner mechanism comprises two switchable inner mechanisms.

46. A system according to claim 44, wherein said at least one inner mechanism comprises an inner tube and said system comprises a third contrast injection port associated with said inner tube.

47. A system according to claim 44, said sheath is bent to facilitate perpendicular positioning of a tip of said sheath against an inner wall of a target blood vessel.

48. A system according to claim 47, said inner mechanism is bent to match said bend in said sheath.

49. A system according to claim 47, comprising a straight guide wire adapted to fit in said sheath and maintain said sheath straight when said sheath is guided to a target area.

50. A system according to claim 44, wherein said at least one inner mechanism comprises a punch.

51. A system according to claim 50, comprising an inner tube having a diameter that varies, along its length between a diameter of said punch and an inner diameter of said sheath.

52. A system according to claim 50, comprising balloon distal of said punch and having a diameter that varies between a diameter of said punch and an inner diameter of said sheath.

53. An anastomotic connector, comprising:
a cylinder-like body; and
at least one set of spikes, coupled to said body by twisting joints.

54. A connector according to claim 53, wherein said spikes are adapted not to penetrate tissue which the spikes contact.

55. A connector according to claim 53, wherein said twisting joints comprise at least one torsion bar.

56. A connector according to claim 53, wherein said twisting joints comprise at least one bend area.

57. A connector according to claim 53, wherein said set of spikes are bent.

58. A connector according to claim 57, wherein said set of spikes are bent at two different locations along the spikes.

59. A connector according to claim 57, wherein each spike comprises two arms that meet at a tip of the spike and are each attached to a different part of said connector.

60. A connector according to claim 59, wherein each arm is attached to a base extension of said connector, by a twisting joint.

61. A connector according to claim 60, wherein said arms and said base extensions define a continuous curve.

62. A fixating guide sheath for insertion into a blood vessel, comprising:
an inner tube; and
an outer tube, slotted near an end thereof, wherein said inner tube is retracted relative to said outer tube, said slotted outer tube flares out to prevent further retraction of said sheath. 63. A sheath according to claim 62, wherein said sheath is bent near said end.
